(19) United States
(12) Patent Application Publication
(10) Pub. No.: US 2024/0219381 A1
(43) Pub. Date: Jul. 4, 2024

(54) METHOD FOR DETERMINING THE AMOUNT OF A THERAPEUTIC ANTIBODY IN THE BRAIN

(71) Applicant: Hoffmann-La Roche Inc., Little Falls, NJ (US)

(72) Inventors: Martin Schaefer, Muenchen (DE); Sylvia Rottach, Antdorf (DE); Gregor Jordan, Gröbenzell (DE); Kay-Gunnar Stubenrauch, Penzberg (DE); Roland Staack, Muenchen (DE); Kevin Brady, Charfield (GB)

(73) Assignee: Hoffmann-La Roche Inc., Little Falls, NJ (US)

(21) Appl. No.: 18/430,887

(22) Filed: Feb. 2, 2024

Related U.S. Application Data
(63) Continuation of application No. 17/136,289, filed on Dec. 29, 2020, now Pat. No. 11,913,945.

(30) Foreign Application Priority Data
Jan. 2, 2020 (EP) 20150135.0

(51) Int. Cl. G01N 33/543; C07K 16/28; C07K 16/40; G01N 33/68

(52) U.S. Cl. CPC G01N 33/543; C07K 16/2803; C07K 16/2881; C07K 16/2887; C07K 16/40; G01N 33/54306; G01N 33/6854; C07K 2317/31; G01N 2800/52

(57) ABSTRACT

Herein is reported a method for determining the concentration of a therapeutic antibody in a tissue of an experimental animal to whom the therapeutic antibody had been administered, wherein the interference from residual blood in a tissue sample of the experimental animal, which is used for determining the concentration of the therapeutic antibody in said tissue, is reduced, wherein the concentration of the therapeutic antibody in the tissue of the experimental animal is calculated with the following formula:

$${C}_{tmAb , t i s s u e} = \frac{{C}_{t mAb , tissue , det .}}{{C}_{tissue , sample}} - \frac{\frac{{C}_{refmAb , tissue , det .}}{{C}_{tissue , sample}}}{{C}_{refmAb , plasma , det .}} * {C}_{tmAb , plasma , det .}$$

wherein C_{tmAb,tissue,det.}=obtained by determining the concentration of the therapeutic antibody in the tissue sample of the experimental animal, C_{tmAb,plasma,det.}=obtained by determining the concentration of the therapeutic antibody in a blood sample of the experimental animal directly prior to taking the tissue sample, C_{refmAb,tissue,det.}=obtained by determining the concentration of the inert reference antibody in the tissue sample of the experimental animal, C_{refmAb,plasma,det.}=obtained by determining the concentration of an inert reference antibody in the blood sample of the experimental animal directly prior to taking the tissue sample, C_{tissue,sample}=obtained by determining the tissue concentration in the tissue sample, whereby the inert reference antibody does not penetrate into said tissue, whereby the inert reference antibody is administered 2 to 10 minutes prior to obtaining the tissue and blood sample.

### CROSS-REFERENCE TO RELATED APPLICATIONS

This application is a continuation of U.S. patent application Ser. No. 17/136,289 filed Dec. 29, 2020 which claims priority to European Patent Application No. EP20150135.0, filed Jan. 2, 2020, all of which are incorporated by reference in its entirety.

### SEQUENCE LISTING

This application contains a Sequence Listing which has been submitted electronically in XML format and is hereby incorporated by reference in its entirety. Said XML copy, created on Jan. 9, 2024, is named P35855-US-2_Seq_List.xml and is 90,288 bytes in size.

### FIELD OF THE INVENTION

The current invention is in the field of immunoassays. More specifically, herein is reported a method for the determination of the amount of a therapeutic antibody in brain tissue, more specifically of the amount of a therapeutic antibody transported across the blood-brain-barrier from the blood into the brain.

### BACKGROUND

For the analysis of therapeutic monoclonal antibodies (tmAbs) in samples of in-vitro or in-vivo origin a respective assay is necessary.

Determining the amount of a therapeutic antibody is generally carried out by determining the amount of said therapeutic antibody in a sample. Therefore, e.g., an immunoassay, such as, ELISA, RIA, protein blot (Western blot) assay, and the like can be used.

The role of antibodies and their receptors in protection against ordered protein assembly in neurodegeneration was reviewed by Katsinelos et al. (Front. Immunol. 10 (2019) A1139). They have outlined that IgG levels are maintained in human serum at around 10 mg/ml. The brain is isolated from serum by the blood-brain barrier (BBB), which is impermeable to large macromolecules including IgG and is bathed in cerebrospinal fluid (CSF), which is produced following the filtration of blood and transport of ions across the choroid plexus. Thus, the resulting concentration of IgG in CSF is around 500- to 1,000-fold lower than in serum.

Hanzatian et al. (mAbs 10 (2018) 765-777) reported that therapeutic monoclonal antibodies and endogenous IgG antibodies show limited uptake into the central nervous system (CNS) due to the blood-brain barrier (BBB), which regulates and controls the selective and specific transport of both exogenous and endogenous materials to the brain. The use of natural transport mechanisms, such as receptor-mediated transcytosis (RMT), to deliver antibody therapeutics into the brain have been studied in rodents and monkeys. After systemic administration of each DVD-Ig, Hanzatian et al. used two independent methods in parallel to observe specific uptake into the brain: An electrochemiluminescent-based sensitive quantitative assay and a semi-quantitative immunohistochemistry technique were used for brain concentration determination and bio distribution/localization in brain, respectively. Significantly enhanced brain uptake and retention was observed for all TfR1 DVD-Ig proteins regardless of the CNS target or the systemic administration route selected. To prepare brain samples for analysis the used C57BL/6N mice were transcardially perfused with cold Dulbecco's phosphate-buffered saline (PBS) containing heparin at a rate of 2 ml/min for 10 min via programmable peristaltic pump.

A comparable approach had been used by Zuchero et al. (Neuron 89 (2016) 70-82; wild-type mice, which were IV injected with the target antibody followed by collection of the whole blood and PBS perfusion) and Janowicz et al. (Nature Sci. Rep. 9 (2019) 9255; P301L tau transgenic pR5 mice, to which Alexa-647-labeled IgG, Fab or scFv had been administered by retro-orbital injection, were perfused following treatment to remove the antibody from their vasculature).

In WO 2018/152359, mice overexpressing human Tau from PS 19 line were used to evaluate target engagement of chimeric IgG anti-tau antibody clones 1C7 and 1A1. Therefore, mice were injected i.v. (at 35 mg/kg) or i.p. (at 50 mg/kg) with a control IgG, chimeric IgG clone 1C7, or chimeric IgG clone 1A1. At 2 or 7 days post-injection, cerebral spinal fluid (CSF) was collected via the cistema magna and visually inspected for potential blood contamination and following transcardial perfusion with ice-cold PBS, brain tissue was removed and snap frozen.

Ayabe, M., et al. reported that an anti-human interleukin-6 receptor (hIL-6R) antibody or control antibody were administered intravenously to tumor-bearing hIL-6R transgenic mice and bovine serum albumin (BSA) was administered intravenously as a marker for residual blood volume in tissues. The lysate samples were treated with immune precipitation using anti-BSA antibody and Protein A magnetic beads followed by tryptic digestion. Each surrogate peptide was analyzed simultaneously by LC/ESI-MS/MS. Corrected tissue concentration was calculated.

Vedeler, et al., reported about immunoglobulins in serum and cerebrospinal fluid from patients with acute Guillain-Barré syndrome (Acta Neurol. Scand. 73 (1986) 388-393.

Shah, et al., reported antibody bio distribution coefficients, especially inferring tissue concentrations of monoclonal antibodies based on the plasma concentrations in several preclinical species and human (MABS, 5 (2013) 297-305).

Lavezzi, et al., reported MPBPK-TMDD models for mAbs, especially alternative models, comparison, and identifiability issues (J. Pharmacokin. Pharmcodyn. 45 (2018) 787-802).

### SUMMARY OF THE INVENTION

Herein is reported a method for the determination of the amount of a therapeutic antibody, which has been transported across the blood-brain-barrier from the blood into the brain of an experimental animal. The amount is preferably determined in a brain lysate sample. The gist of the invention lies in the additional application of an inert antibody, which is not transported across the blood-brain-barrier, shortly before obtaining the brain sample in which the amount of the therapeutic antibody transported across the blood-brain-barrier has to be determined. By applying the inert antibody, a correction value for therapeutic antibody present in residual blood in the brain sample is obtained. This residual blood-derived amount is used to correct the determined amount for non-brain-located antibody. A determination without correction would determine the total amount of therapeutic antibody in the sample, i.e. the amount transported across the blood-brain-barrier into the brain and the amount in residual blood in the sample. The amount of therapeutic antibody in residual blood is not neglectable, as only about 0.1% of the antibody in the blood will pass the blood-brain-barrier. Thus, the concentration of the therapeutic antibody in the blood exceeds the concentration of the therapeutic antibody in the brain by at least two and up to three orders of magnitude. Thereby the results obtained are too high if not corrected with a method according to the current invention.

The current invention is based, at least in part, on the finding that for a robust and correct determination of the amount in brain lysates of a therapeutic antibody transported across the blood-brain-barrier into the brain a correction, i.e. reduction, with the amount of therapeutic antibody in residual blood in the brain lysate sample has to be made.

The current invention is based, at least in part, on the finding that the amount of residual blood in a brain lysate can be determined by applying a correction antibody shortly before the brain sample is taken. It has been found that it is especially advantageous to use as reference antibody an antibody that is not specifically binding to any target in the experimental animal from which the brain sample is obtained, most preferably a human germline antibody.

One aspect of the invention is a method/assay for determining the concentration of a therapeutic antibody in a tissue of an experimental animal, whereby the tissue has a barrier to the blood circulation of said animal and whereby the therapeutic antibody had been administered to said experimental animal, wherein the interference from residual blood in a tissue sample of the experimental animal, which is used for determining the concentration of the therapeutic antibody in said tissue, is reduced, the method comprising the following steps
i) determining the concentration of the therapeutic antibody in a blood sample of the experimental animal,
ii) determining the concentration of the therapeutic antibody in the tissue sample of the experimental animal,
iii) determining the concentration of an inert reference antibody in the blood sample of the experimental animal,
iv) determining the concentration of the inert reference antibody in the tissue sample of the experimental animal,
v) determining the tissue concentration in the tissue sample,
and determining the concentration of the therapeutic antibody in the tissue of the experimental animal with the following formula:

$${C}_{tmAb , tissue} = \frac{{c}_{tmAb , tissue , det .}}{{c}_{tissue , sample}} - \frac{\frac{{c}_{refmAb , tissue , det .}}{{c}_{tissue , sample}}}{{c}_{refmAb , plasma . det .}} * {C}_{tmAb , plasma , det .}$$

with
   C_{tmAb,plasma,det.}=concentration of the therapeutic antibody of i)
   C_{tmAb,tissue,det.}=concentration of the therapeutic antibody of ii)
   C_{refmAb,tissue,det.}=concentration of the inert reference antibody of iii)
   C_{refmAb,plasma,det.}=concentration of the inert reference antibody of iv)
   C_{tissue,sample}=tissue concentration of v)
whereby the inert reference antibody does not cross said barrier between the tissue and the blood circulation,
whereby the inert reference antibody had been administered i) either together with the therapeutic antibody in case the sample is to be taken within 5 minutes after the administration of the therapeutic antibody, or ii) 2 to 10 minutes prior to taking the tissue sample.

The same aspect in an alternative wording is, a method for determining the concentration of a therapeutic antibody in a tissue of an experimental animal to whom the therapeutic antibody had been administered, wherein the interference from residual blood in a tissue sample of the experimental animal, which is used for determining the concentration of the therapeutic antibody in said tissue, is reduced,
wherein the concentration of the therapeutic antibody in the tissue of the experimental animal is calculated with the following formula:

$${C}_{tmAb , tissue} = \frac{{c}_{tmAb , tissue , det .}}{{c}_{tissue , sample}} - \frac{\frac{{c}_{refmAb , tissue , det .}}{{c}_{tissue , sample}}}{{c}_{refmAb , plasma . det .}} * {C}_{tmAb , plasma , det .}$$

wherein
   C_{tmAb,tissue,det.}=obtained by determining the concentration of the therapeutic antibody in the tissue sample of the experimental animal,
   C_{tmAb,plasma,det.}=obtained by determining the concentration of the therapeutic antibody in a blood sample of the experimental animal,
   C_{refmAb,tissue,det.}=obtained by determining the concentration of the inert reference antibody in the tissue sample of the experimental animal,
   C_{refmAb,plasma,det.}=obtained by determining the concentration of an inert reference antibody in the blood sample of the experimental animal,
   C_{tissue,sample}=obtained by determining the tissue concentration in the tissue sample,
whereby the inert reference antibody does not penetrate into said tissue,
whereby the inert reference antibody is administered 2 to 10 minutes prior to obtaining the tissue sample.

The following are all individual embodiment of each and any aspects of the invention. Thus, all and any possible permutation of embodiments is disclosed with respect to any individual aspect according to the invention,

In one embodiment, the blood sample is taken at most 5 minutes prior to the tissue sample. In one embodiment, the blood sample is taken prior to the tissue sample. In one embodiment, the blood sample is taken together or at the same time as the tissue sample.

In one embodiment, the tissue is either brain tissue and the therapeutic antibody can cross the blood-brain-barrier or ocular tissue and the therapeutic antibody can cross the blood-ocular-barrier.

One aspect of the invention is a method/assay for determining the concentration of a therapeutic antibody in brain tissue or a brain tissue sample of an experimental animal, whereby the brain tissue has a barrier to the blood circulation of said animal and whereby the therapeutic antibody had been administered to said experimental animal, wherein the interference from residual blood in a brain tissue sample of the experimental animal, which is used for determining the concentration of the therapeutic antibody in said brain tissue, is reduced, the method comprising the following steps
i) determining the concentration of the therapeutic antibody in a blood sample of the experimental animal,
ii) determining the concentration of the therapeutic antibody in the brain tissue sample of the experimental animal,
iii) determining the concentration of an inert reference antibody in the blood sample of the experimental animal,
iv) determining the concentration of the inert reference antibody in the brain tissue sample of the experimental animal,
v) determining the brain tissue concentration in the tissue sample,
and determining the concentration of the therapeutic antibody in the brain tissue or the brain tissue sample of the experimental animal with the following formula:

$${C}_{tmAb , tissue} = \frac{{c}_{tmAb , tissue , det .}}{{c}_{tissue , sample}} - \frac{\frac{{c}_{refmAb , tissue , det .}}{{c}_{tissue , sample}}}{{c}_{refmAb , plasma . det .}} * {C}_{tmAb , plasma , det .}$$

with
   C_{tmAb,plasma,det.}=concentration of the therapeutic antibody of i)
   C_{tmAb,tissue,det.}=concentration of the therapeutic antibody of ii)
   C_{refmAb,tissue,det.}=concentration of the inert reference antibody of iii)
   C_{refmAb,plasma,det.}=concentration of the inert reference antibody of iv)
   C_{tissue,sample}=tissue concentration of v)
whereby the inert reference antibody does not cross said blood-brain-barrier between the brain tissue and the blood circulation,
whereby the inert reference antibody had been administered i) either together with the therapeutic antibody in case the brain tissue sample is to be taken within 5 minutes after the administration of the therapeutic antibody, or ii) 2 to 10 minutes prior to taking the brain tissue sample.

The same aspect in an alternative wording is, a method for determining the concentration of a therapeutic antibody in brain tissue or a brain tissue sample of an experimental animal to whom the therapeutic antibody had been administered, wherein the interference from residual blood in the brain tissue sample of the experimental animal, which is used for determining the concentration of the therapeutic antibody in said brain tissue, is reduced,
wherein the concentration of the therapeutic antibody in the brain tissue of the experimental animal is calculated with the following formula:

$${C}_{tmAb , tissue} = \frac{{c}_{tmAb , tissue , det .}}{{c}_{tissue , sample}} - \frac{\frac{{c}_{refmAb , tissue , det .}}{{c}_{tissue , sample}}}{{c}_{refmAb , plasma . det .}} * {C}_{tmAb , plasma , det .}$$

wherein
   C_{tmAb,tissue,det.}=obtained by determining the concentration of the therapeutic antibody in the brain tissue sample of the experimental animal,
   C_{tmAb,plasma,det.}=obtained by determining the concentration of the therapeutic antibody in a blood sample of the experimental animal,
   C_{refmAb,tissue,det.}=obtained by determining the concentration of the inert reference antibody in the brain tissue sample of the experimental animal,
   C_{refmAb,plasma,det.}=obtained by determining the concentration of an inert reference antibody in the blood sample of the experimental animal,
   C_{tissue,sample}=obtained by determining the brain tissue concentration in the brain tissue sample,
whereby the inert reference antibody does not penetrate into said brain tissue,
whereby the inert reference antibody is administered 2 to 10 minutes prior to obtaining the brain tissue sample.

In one embodiment, the therapeutic antibody is a bispecific antibody.

In one embodiment, the therapeutic antibody is specifically binding to human transferrin receptor and a brain target.

In one embodiment, the brain target is human CD20 or human Abeta or human alpha-synuclein or human tau or human glucocerebrosidase or human lingo-1 or human huntingtin.

In one embodiment, the experimental animal is selected from mouse, rat, rabbit, dog, sheep, ape, and monkey.

In one embodiment, the experimental animal is a non-human experimental animal with a body weight of more than 100 g and less than 15 kg.

In one embodiment, the experimental animal is a cynomolgus monkey.

In one embodiment, the inert reference antibody is a human germline antibody.

In one embodiment, the inert reference antibody is DP47GS. In one embodiment, the inert reference antibody comprises a heavy chain variable domain of SEQ ID NO: 67 and a light chain variable domain of SEQ ID NO: 68. In one embodiment, the inert reference antibody comprises a heavy chain of SEQ ID NO: 69 and a light chain of SEQ ID NO: 70.

In one embodiment, the inert reference antibody does not cross said barrier in detectable amounts within 15 minutes after its application.

In one embodiment, the inert reference antibody does not cross said barrier in detectable amounts within 10 minutes after its application.

In one embodiment, the inert antibody is administered 5 to 10 minutes prior to taking the tissue sample.

In one embodiment, the tissue is perfused with an aqueous solution directly after taking the blood sample and prior to taking the tissue sample.

In one embodiment, the determining of the concentrations is by a bridging ELISA.

### DETAILED DESCRIPTION OF EMBODIMENTS OF THE INVENTION

Herein is reported a method for the determination of the amount of a therapeutic antibody, which has been transported across the blood-brain-barrier from the blood into the brain of an experimental animal. The amount is preferably determined in a brain lysate sample. The gist of the invention lies in the additional application of an inert antibody, which is not transported across the blood-brain-barrier, shortly before obtaining the brain sample in which the amount of the therapeutic antibody transported across the blood-brain-barrier has to be determined. By applying the inert antibody, a correction value for therapeutic antibody present in residual blood in the brain sample is obtained. This residual blood-derived amount is used to correct the determined amount for non-brain-located antibody. A determination without correction would determine the total amount of therapeutic antibody in the sample, i.e. the amount transported across the blood-brain-barrier into the brain and the amount in residual blood in the sample. The amount of therapeutic antibody in residual blood is not neglectable, as only about 0.1% of the antibody in the blood will pass the blood-brain-barrier. Thus, the concentration of the therapeutic antibody in the blood exceeds the concentration of the therapeutic antibody in the brain by at least two and up to three orders of magnitude. Thereby the results obtained are too high if not corrected with a method according to the current invention.

The current invention is based, at least in part, on the finding that for a robust and correct determination of the amount in brain lysates of a therapeutic antibody transported across the blood-brain-barrier into the brain a correction, i.e. reduction, with the amount of therapeutic antibody in residual blood in the brain lysate sample has to be made.

The current invention is based, at least in part, on the finding that the amount of residual blood in a brain lysate can be determined by applying a correction antibody shortly before the brain sample is taken. It has been found that it is especially advantageous to use as reference antibody an antibody that is not specifically binding to any target in the experimental animal from which the brain sample is obtained, most preferably a human germline antibody.

One aspect of the invention is a method/assay for determining the concentration of a therapeutic antibody in a tissue of an experimental animal, whereby the tissue has a barrier to the blood circulation of said animal and whereby the therapeutic antibody had been administered to said experimental animal, wherein the interference from residual blood in a tissue sample of the experimental animal, which is used for determining the concentration of the therapeutic antibody in said tissue, is reduced, the method comprising the following steps
i) determining the concentration of the therapeutic antibody in a blood serum sample of the experimental animal,
ii) determining the concentration of the therapeutic antibody in the tissue sample of the experimental animal,
iii) determining the concentration of an inert reference antibody in the blood serum sample of the experimental animal,
iv) determining the concentration of the inert reference antibody in the tissue sample of the experimental animal,
v) determining the tissue concentration in the tissue sample,
and determining the concentration of the therapeutic antibody in the tissue of the experimental animal with the following formula:

$${C}_{tmAb , tissue} = \frac{{c}_{tmAb , tissue , det .}}{{c}_{tissue , sample}} - \frac{\frac{{c}_{refmAb , tissue , det .}}{{c}_{tissue , sample}}}{{c}_{refmAb , plasma . det .}} * {C}_{tmAb , plasma , det .}$$

with
   C_{tmAb,plasma,det.}=concentration of the therapeutic antibody of i)
   C_{tmAb,tissue,det.}=concentration of the therapeutic antibody of ii)
   C_{refmAb,tissue,det.}=concentration of the inert reference antibody of iii)
   C_{refmAb,plasma,det.}=concentration of the inert reference antibody of iv)
   C_{tissue,sample}=tissue concentration of v)
whereby the inert reference antibody does not cross said barrier between the tissue and the blood circulation,
whereby the inert reference antibody had been administered i) either together with the therapeutic antibody in case the sample is to be taken within 5 minutes after the administration of the therapeutic antibody, or ii) 2 to 10 minutes prior to taking the tissue sample,
whereby the blood sample is taken together/directly prior/at the same time as the tissue sample.

### Definitions

The knobs into holes dimerization modules and their use in antibody engineering are described in Carter P.; Ridgway J. B. B.; Presta L. G.: Immunotechnology, Volume 2, Number 1, February 1996, pp. 73-73(1).

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, MD (1991).

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, MD (1991) and is referred to as “numbering according to Kabat” herein. Specifically, the Kabat numbering system (sec pages 647-660) of Kabat, et al., Sequences of Proteins of Immunological Interest, 5th ed., Public Health Service, National Institutes of Health, Bethesda, MD (1991) is used for the light chain constant domain CL of kappa and lambda isotype, and the Kabat EU index numbering system (see pages 661-723) is used for the constant heavy chain domains (CH1, Hinge, CH2 and CH3, which is herein further clarified by referring to “numbering according to Kabat EU index” in this case).

The term “about” denotes a range of +/−20% of the thereafter following numerical value. In one embodiment, the term about denotes a range of +/−10% of the thereafter-following numerical value. In one embodiment, the term about denotes a range of +/−5% of the thereafter-following numerical value.

The term “antibody-dependent cellular cytotoxicity (ADCC)” is a function mediated by Fc receptor binding and refers to lysis of target cells by an antibody as reported herein in the presence of effector cells. ADCC is measured in one embodiment by the treatment of a preparation of CD19 expressing crythroid cells (e.g. K562 cells expressing recombinant human CD19) with an antibody as reported herein in the presence of effector cells such as freshly isolated PBMC (peripheral blood mononuclear cells) or purified effector cells from buffy coats, like monocytes or NK (natural killer) cells. Target cells are labeled with 51Cr and subsequently incubated with the antibody. The labeled cells are incubated with effector cells and the supernatant is analyzed for released 51Cr. Controls include the incubation of the target endothelial cells with effector cells but without the antibody. The capacity of the antibody to induce the initial steps mediating ADCC is investigated by measuring their binding to Fcγ receptors expressing cells, such as cells, recombinantly expressing FcγRI and/or FcγRIIA or NK cells (expressing essentially FcγRIIIA). In one preferred embodiment, binding to FcγR on NK cells is measured.

The term “amplifier” denotes an entity or process that enhances the signal in a detection method, such as an ELISA (e.g., an enzymatic amplifier used in an ELISA).

The terms “anti-human A-beta antibody” and “an antibody specifically binding to human A-beta” refer to an antibody that is capable of binding the human A-beta peptide with sufficient affinity such that the antibody is useful as a diagnostic and/or therapeutic agent in targeting A-beta peptide.

It is of note that human A-beta has several naturally occurring forms, whereby the human forms are referred to as Aβ39, Aβ40, Aβ41, Aβ42 and Aβ43. The most prominent form, Aβ42, has the amino acid sequence of SEQ ID NO: 01. In Aβ41, Aβ40, Aβ39, the C-terminal amino acids A, IA and VIA are missing, respectively. In the Aβ43 form, an additional threonine residue is comprised at the C-terminus of SEQ ID NO: 01 (33106).

Thus, the term also encompasses antibodies that bind to a shortened fragment of the human A-beta polypeptide.

The term “antibody” herein is used in the broadest sense and encompasses various antibody structures, including but not limited to monoclonal antibodies, polyclonal antibodies, or multispecific antibodies (e.g., bispecific antibodies).

An antibody in general comprises two so called light chain polypeptides (light chain) and two so called heavy chain polypeptides (heavy chain). Each of the heavy and light chain polypeptides contains a variable domain (variable region) (generally the amino terminal portion of the polypeptide chain) comprising binding regions that are able to interact with an antigen. Each of the heavy and light chain polypeptides comprises a constant region (generally the carboxyl terminal portion). The constant region of the heavy chain mediates the binding of the antibody i) to cells bearing a Fc gamma receptor (FcγR), such as phagocytic cells, or ii) to cells bearing the neonatal Fc receptor (FcRn) also known as Brambell receptor. It also mediates the binding to some factors including factors of the classical complement system such as component (C1q). The constant domains of an antibody heavy chain comprise the CH1-domain, the CH2-domain and the CH3-domain, whereas the light chain comprises only one constant domain, CL, which can be of the kappa isotype or the lambda isotype.

The variable domain of an immunoglobulin's light or heavy chain in turn comprises different segments, i.e. four framework regions (FR) and three hypervariable regions (HVR).

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The “blood-brain-barrier” or “BBB” refers to the physiological barrier between the peripheral circulation and the brain and spinal cord, which is formed by tight junctions within the brain capillary endothelial plasma membranes, creating a tight barrier that, restricts the transport of molecules into the brain, even very small molecules such as urea (60 Daltons). The BBB within the brain, the blood-spinal-cord barrier within the spinal cord, and the blood-retinal-barrier within the retina are contiguous capillary barriers within the CNS, and are herein collectively referred to an the blood-brain-barrier or BBB. The BBB also encompasses the blood-CSF barrier (choroid plexus) where the barrier is comprised of ependymal cells rather than capillary endothelial cells.

A “blood-brain-barrier receptor” (abbreviated “BBBR” herein) is an extracellular membrane-linked receptor protein expressed on brain endothelial cells which is capable of transporting molecules across the BBB or be used to transport exogenous administrated molecules. Examples of BBBR herein include: transferrin receptor (TfR), insulin receptor, insulin-like growth factor receptor (IGF-R), low density lipoprotein receptors including without limitation low density lipoprotein receptor-related protein 1 (LRP1) and low density lipoprotein receptor-related protein 8 (LRP8), and heparin-binding epidermal growth factor-like growth factor (HB-EGF). One preferred BBBR is transferrin receptor (TfR).

The term “brain effector entity” denotes a molecule that is to be transported to the brain across the BBB. The effector entity typically has a characteristic therapeutic activity that is desired to be delivered to the brain. Effector entities include neurologically disorder drugs and cytotoxic agents such as e.g. polypeptides and antibodies, in particular monoclonal antibodies or fragments thereof directed to a brain target.

The term “capture antibody” denotes an antibody that is used in a sandwich ELISA format to bind (i.e., capture) a target substance present in a sample for detection. A second antibody (i.e., the detection antibody) then binds to the captured target and allows detection of the antibody-target-antibody-complex (forming a “sandwich” comprised of antibody-target-antibody).

The “central nervous system” or “CNS” refers to the complex of nerve tissues that control bodily function, and includes the brain and spinal cord.

The terms “CNS antigen” and “brain target” denote an antigen and/or molecule expressed in the CNS, including the brain, which can be targeted with an antibody or small molecule. Examples of such antigen and/or molecule include, without limitation: beta-secretase 1 (BACE1), amyloid beta (Abeta), epidermal growth factor receptor (EGFR), human epidermal growth factor receptor 2 (HER2), Tau, apolipoprotein E4 (ApoE4), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), glucocerebrosidase and caspase 6.

A “conjugate” is fusion protein of the present invention conjugated to one or more heterologous molecule(s), including but not limited to a label, neurological disorder drug or cytotoxic agent.

The term “detection antibody” denotes an antibody, which carries a means for visualization or quantitation. Such a means is typically an enzyme (catalyzing a colored or fluorescent reaction product following the addition of a suitable substrate), such as, e.g., horseradish peroxidase, urease, alkaline phosphatase, glucoamylase and β-galactosidase. In some embodiments, the detection antibody is directed against the antigen of interest. In some embodiments, the detection antibody is an anti-species antibody. In some embodiments, the detection antibody is conjugated to a detectable label such as biotin, a fluorescent marker, or a radioisotope, and is detected and/or quantitated using this label.

The term “detection reagent” denotes a reagent, which permits the detection and/or quantitation of an antibody, bound to an antigen. In some embodiments, the detection reagent is a colorimetric substrate for an enzyme that has been conjugated to an antibody. Addition of a suitable substrate to the antibody-enzyme conjugate results in the production of a colorimetric or fluorimetric signal (e.g., following the binding of the conjugated antibody to the antigen of interest). This definition also encompasses the use of biotin and avidin-based compounds (e.g., including but not limited to neutravidin and streptavidin) as part of the detection system.

The term “directly after” as used herein denotes the time span between taking a first sample and a second sample, which only encompasses the change of the sampling device and the actual time for taking the sample. In one embodiment, the term directly after denotes a time period of 5 minutes or less, in a further embodiment, of 3 minutes or less, in one preferred embodiment, of 2 minutes or less.

“Effector functions” refer to those biological activities attributable to the Fc region of an antibody, which vary with the antibody class. Examples of antibody effector functions include C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

Fc receptor binding dependent effector functions can be mediated by the interaction of the Fc-region of an antibody with Fc receptors (FcRs), which are specialized cell surface receptors on hematopoietic cells. Fc receptors belong to the immunoglobulin superfamily, and have been shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC) (see e.g. Van de Winkel, J. G. and Anderson, C. L., J. Leukoc. Biol. 49 (1991) 511-524). FcRs are defined by their specificity for immunoglobulin isotypes: Fc receptors for IgG antibodies are referred to as FcγR. Fc receptor binding is described e.g. in Ravetch, J. V. and Kinet, J. P., Annu. Rev. Immunol. 9 (1991) 457-492; Capel, P. J., et al., Immunomethods 4 (1994) 25-34; de Haas, M., et al., J. Lab. Clin. Med. 126 (1995) 330-341; and Gessner, J. E., et al., Ann. Hematol. 76 (1998) 231-248.

Cross-linking of receptors for the Fc-region of IgG antibodies (FcγR) triggers a wide variety of effector functions including phagocytosis, antibody-dependent cellular cytotoxicity, and release of inflammatory mediators, as well as immune complex clearance and regulation of antibody production. In humans, three classes of FcγR have been characterized, which are:
FcγRI (CD64) binds monomeric IgG with high affinity and is expressed on macrophages, monocytes, neutrophils and cosinophils. Modification in the Fc-region IgG at least at one of the amino acid residues E233-G236, P238, D265, N297, A327 and P329 (numbering according to EU index of Kabat) reduce binding to FcγRI. IgG2 residues at positions 233-236, substituted into IgG1 and IgG4, reduced binding to FcγRI by 103-fold and eliminated the human monocyte response to antibody-sensitized red blood cells (Armour, K. L., et al., Eur. J. Immunol. 29 (1999) 2613-2624).
FcγRII (CD32) binds complexed IgG with medium to low affinity and is widely expressed. This receptor can be divided into two sub-types, FcγRIIA and FcγRIIB. FcγRIIA is found on many cells involved in killing (e.g. macrophages, monocytes, neutrophils) and seems able to activate the killing process. FcγRIIB seems to play a role in inhibitory processes and is found on B-cells, macrophages and on mast cells and cosinophils. On B-cells, it seems to function to suppress further immunoglobulin production and isotype switching to, for example, the IgE class. On macrophages, FcγRIIB acts to inhibit phagocytosis as mediated through FcγRIIA. On eosinophils and mast cells, the B-form may help to suppress activation of these cells through IgE binding to its separate receptor. Reduced binding for FcγRIIA is found e.g. for antibodies comprising an IgG Fc-region with mutations at least at one of the amino acid residues E233-G236, P238, D265, N297, A327, P329, D270, Q295, A327, R292, and K41 (numbering according to EU index of Kabat).
FcγRIII (CD16) binds IgG with medium to low affinity and exists as two types. FcγRIIIA is found on NK cells, macrophages, eosinophils and some monocytes and T cells and mediates ADCC. FcγRIIIB is highly expressed on neutrophils. Reduced binding to FcγRIIIA is found e.g. for antibodies comprising an IgG Fc-region with mutation at least at one of the amino acid residues E233-G236, P238, D265, N297, A327, P329, D270, Q295, A327, S239, E269, E293, Y296, V303, A327, K338 and D376 (numbering according to EU index of Kabat).

Mapping of the binding sites on human IgG1 for Fc receptors, the above mentioned mutation sites and methods for measuring binding to FcγRI and FcγRIIA are described in Shields, R. L., et al. J. Biol. Chem. 276 (2001) 6591-6604.

An “effective amount” of an agent, e.g., a pharmaceutical formulation, refers to an amount effective, at dosages and for periods of time necessary, to achieve the desired therapeutic or prophylactic result.

The term “ELISA” denotes an enzyme-linked immunosorbent assay. Different ELISA formats and applications are known in the art (see, e.g., Crowther, “Enzyme-Linked Immunosorbent Assay (ELISA),” in Molecular Biomethods Handbook, Rapley et al. [eds.], pp. 595-617, Humana Press, Inc., Totowa, NJ (1998); Harlow and Lane (eds.), Antibodies: A Laboratory Manual, Cold Spring Harbor Laboratory Press (1988); Ausubel et al. (eds.), Current Protocols in Molecular Biology, Ch. 11, John Wiley & Sons, Inc., New York (1994)).

One specific ELISA format is a so-called “direct ELISA”. In this ELISA format a target, e.g. a polypeptide, present in a sample is detected. In a direct ELISA the sample, containing the target, is brought in contact with a solid phase, such as e.g., stationary or immobilized support (e.g., a microtiter plate well). The target, if present in the sample, becomes immobilized to the solid phase, and is thereafter detected directly using an enzyme-conjugated detection molecule. If the target is an antigen the detection molecule is an antibody specific for the antigen, or if the target is an antibody specific for an antigen the detection molecule is an enzyme-conjugated antibody specific for the antigen.

Another specific ELISA format is a so-called “indirect ELISA”. In this ELISA format, an antigen (or an antibody) is immobilized to a solid phase (e.g., a microtiter plate well). Thereafter an antigen-specific antibody (or antigen) is added followed by the addition of a detection antibody specific for the antibody that specifically binds the antigen. This detection antibody can be a “species-specific” antibody (e.g., a goat anti-rabbit antibody).

Another specific ELISA format is a so-called “sandwich ELISA”. In this format the antigen is immobilized on a solid phase (e.g., a microtiter plate well) via capture by an antibody specifically binding to the antigen (i.e., a capture antibody), which is (covalently or via a specific binding pair) immobilized on the solid phase. Generally, a sample comprising the antigen is added to the solid phase, followed by washing. If the antigen of interest is present in the sample, it is bound by the capture antibody to the solid phase.

The above-specified ELISA formats can be combined. A sandwich ELISA can be a “direct sandwich ELISA”, wherein the captured antigen is detected directly by using an enzyme-conjugated antibody directed against the antigen. A sandwich ELISA can be an “indirect sandwich ELISA”, wherein the captured antigen is detected indirectly by using an antibody directed against the antigen, which is then detected by another enzyme-conjugated antibody which binds the antigen-specific antibody either directly or via an attached label. With a reporter reagent, the third antibody is detected.

The term “Fc receptor” as used herein refers to activation receptors characterized by the presence of a cytoplasmic ITAM sequence associated with the receptor (see e.g. Ravetch, J. V. and Bolland, S., Annu. Rev. Immunol. 19 (2001) 275-290). Such receptors are FcγRI, FcγRIIA and FcγRIIIA. The term “no binding of FcγR” denotes that at an antibody concentration of 10 μg/ml the binding of an antibody as reported herein to NK cells is 10% or less of the binding found for anti-OX40L antibody LC.001 as reported in WO 2006/029879.

While IgG4 shows reduced FcR binding, antibodies of other IgG subclasses show strong binding. However, Pro238, Asp265, Asp270, Asn297 (loss of Fc carbohydrate), Pro329 and 234, 235, 236 and 237 Ile253, Ser254, Lys288, Thr307, Gln311, Asn434, and His435 are residues which provide if altered also reduce FcR binding (Shields, R. L., et al. J. Biol. Chem. 276 (2001) 6591-6604; Lund, J., et al., FASEB J. 9 (1995) 115-119; Morgan, A., et al., Immunology 86 (1995) 319-324; and EP 0 307 434). In one embodiment, the antibody as reported herein is of IgG1 or IgG2 subclass and comprises the mutation PVA236, GLPSS331, and/or L234A/L235A. In one embodiment, the antibody as reported herein is of IgG4 subclass and comprises the mutation L235E. In one embodiment, the antibody further comprises the mutation S228P.

The term “Fc-region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat, E. A. et al., Sequences of Proteins of Immunological Interest, 5th ed., Public Health Service, National Institutes of Health, Bethesda, MD (1991), NIH Publication 91-3242.

The antibodies as reported herein comprise as Fc-region, in one embodiment, an Fc-region derived from human origin. In one embodiment, the Fc-region comprises all parts of the human constant region. The Fc-region of an antibody is directly involved in complement activation, C1q binding, C3 activation and Fc receptor binding. While the influence of an antibody on the complement system is dependent on certain conditions, binding to C1q is caused by defined binding sites in the Fc-region. Such binding sites are known in the state of the art and described e.g. by Lukas, T. J., et al., J. Immunol. 127 (1981) 2555-2560; Brunhouse, R., and Cebra, J. J., Mol. Immunol. 16 (1979) 907-917; Burton, D. R., et al., Nature 288 (1980) 338-344; Thommesen, J. E., et al., Mol. Immunol. 37 (2000) 995-1004; Idusogic, E. E., et al., J. Immunol. 164 (2000) 4178-4184; Hezarch, M., et al., J. Virol. 75 (2001) 12161-12168; Morgan, A., et al., Immunology 86 (1995) 319-324; and EP 0 307 434. Such binding sites are e.g. L234, L235, D270, N297, E318, K320, K322, P331 and P329 (numbering according to EU index of Kabat; Unless otherwise specified herein, numbering of amino acid residues in the Fc-region or constant region is according to the EU numbering system, also called the EU index, as described in Kabat, E. A. et al., Sequences of Proteins of Immunological Interest, 5th ed., Public Health Service, National Institutes of Health, Bethesda, MD (1991), NIH Publication 91-3242). Antibodies of subclass IgG1, IgG2 and IgG3 usually show complement activation, C1q binding and C3 activation, whereas IgG4 do not activate the complement system, do not bind C1q and do not activate C3. An “Fc-region of an antibody” is a term well known to the skilled artisan and defined on the basis of papain cleavage of antibodies. In one embodiment, the Fc-region is a human Fc-region. In one embodiment, the Fc-region is of the human IgG4 subclass comprising the mutations S228P and/or L235E (numbering according to EU index of Kabat). In one embodiment, the Fc-region is of the human IgG1 subclass comprising the mutations L234A and L235A (numbering according to EU index of Kabat).

The terms “full length antibody”, “intact antibody”, and “whole antibody” are used herein interchangeably to refer to an antibody having a structure substantially similar to a native antibody structure, i.e. comprising two light chains and two heavy chains.

A “human antibody” is one, which possesses an amino acid sequence, which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “in-vitro” denotes either an artificial environment as such or that a process or reaction is performed within such an artificial environment.

The term “in-vivo” denotes the natural environment (e.g., an animal or a cell) of a compound or that a process or reaction is performed within its natural environment.

The term “immunoassay” denotes any technique that utilizes specifically binding molecules, such as antibodies, to capture and/or detect a specific target for qualitative or quantitative analysis. In general, an immunoassay is characterized by the following steps: 1) immobilization or capture of the analyte and 2) detection and measuring the analyte. The analyte can be captured, i.e. bound, on any solid surface, such as e.g. a membrane, plastic plate, or some other solid surface.

The term “linker” denotes a chemical linker or a single chain peptidic linker that covalently connects different entities of the blood-brain-barrier shuttle module and/or the fusion polypeptide and/or the conjugate as reported herein. The linker connects for example the brain effector entity to the monovalent binding entity. For example, if the monovalent binding entity comprises a CH2-CH3 Ig entity and a scFab directed to the blood-brain-barrier-receptor, then the linker conjugates the scFab to the C-terminal end of the CH3-CH2 Ig entity. The linker conjugating the brain effector entity to the monovalent binding entity (first linker) and the linker connecting the scFab to the C-terminal end of the CH2-CH3 Ig domain (second linker) can be the same or different.

Single chain peptidic linkers, comprising of from one to twenty amino acid residues joined by peptide bonds, can be used. In certain embodiments, the amino acids are selected from the twenty naturally occurring amino acids. In certain other embodiments, one or more of the amino acids are selected from glycine, alanine, proline, asparagine, glutamine and lysine. In other embodiments, the linker is a chemical linker. In certain embodiments, the linker is a single chain peptidic linker with an amino acid sequence with a length of at least 25 amino acid residues, in one preferred embodiment, with a length of 32 to 50 amino acid residues. In one embodiment, the peptidic linker is a (GxS)n linker with G=glycine, S=serine, (x=3, n=8, 9 or 10) or (x=4 and n=6, 7 or 8), in one embodiment, with x=4, n=6 or 7, in one preferred embodiment, with x=4, n=7. In one embodiment, the linker is (G4S)4 (SEQ ID NO: 02). In one embodiment, the linker is (G4S)6G2 (SEQ ID NO: 03).

Conjugation may be performed using a variety of chemical linkers. For example, the monovalent binding entity or the fusion polypeptide and the brain effector entity may be conjugated using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). The linker may be a “cleavable linker” facilitating release of the effector entity upon delivery to the brain. For example, an acid-labile linker, peptidase-sensitive linker, photo-labile linker, dimethyl linker or disulfide-containing linker (Chari et al, Cancer Res. 52 (1992) 127-131; U.S. Pat. No. 5,208,020) may be used.

Covalent conjugation can either be direct or via a linker. In certain embodiments, direct conjugation is by construction of a polypeptide fusion (i.e. by genetic fusion of the two genes encoding the monovalent binding entity towards the BBBR and effector entity and expressed as a single polypeptide (chain)). In certain embodiments, direct conjugation is by formation of a covalent bond between a reactive group on one of the two portions of the monovalent binding entity against the BBBR and a corresponding group or acceptor on the brain effector entity. In certain embodiments, direct conjugation is by modification (i.e. genetic modification) of one of the two molecules to be conjugated to include a reactive group (as non-limiting examples, a sulfhydryl group or a carboxyl group) that forms a covalent attachment to the other molecule to be conjugated under appropriate conditions. As one non-limiting example, a molecule (i.e. an amino acid) with a desired reactive group (i.e. a cysteine residue) may be introduced into, e.g., the monovalent binding entity towards the BBBR antibody and a disulfide bond formed with the neurological therapeutic antibody. Methods for covalent conjugation of nucleic acids to proteins are also known in the art (i.e., photo-crosslinking, sec, e.g., Zatsepin et al. Russ. Chem. Rev. 74 (2005) 77-95). Conjugation may also be performed using a variety of linkers. For example, a monovalent binding entity and a effector entity may be conjugated using a variety of bifunctional protein coupling agents such as N-succinimidyl-3-(2-pyridyldithio) propionate (SPDP), succinimidyl-4-(N-malcimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Peptidic linkers, comprised of from one to twenty amino acid residues joined by peptide bonds, may also be used. In certain such embodiments, the amino acid residues are selected from the twenty naturally occurring amino acids. In certain other such embodiments, one or more of the amino acid residues are selected from glycine, alanine, proline, asparagine, glutamine and lysine. The linker may be a “cleavable linker” facilitating release of the effector entity upon delivery to the brain. For example, an acid-labile linker, peptidase-sensitive linker, photo-labile linker, dimethyl linker or disulfide-containing linker (Chari et al, Cancer Res. 52 (1992) 127-131; U.S. Pat. No. 5,208,020) may be used.

The term “monoclonal antibody” as used herein refers to an antibody obtained from a population of substantially homogeneous antibodies, i.e., the individual antibodies comprising the population are identical, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “monovalent binding entity” denotes a molecule able to bind specifically and in a monovalent binding mode to a BBBR. The blood brain shuttle module and/or conjugate as reported herein are characterized by the presence of a single unit of a monovalent binding entity i.e. the blood brain shuttle module and/or conjugate of the present invention comprise exactly one unit of the monovalent binding entity. The monovalent binding entity includes but is not limited to polypeptides, full length antibodies, antibody fragments including Fab, Fab′, Fv fragments, single-chain antibody molecules such as e.g. single chain Fab, scFv. The monovalent binding entity can for example be a scaffold protein engineered using state of the art technologies like phage display or immunization. The monovalent binding entity can also be a polypeptide. In certain embodiments, the monovalent binding entity comprises a CH2-CH3 Ig domain and a single chain Fab (scFab) directed to a blood-brain-barrier-receptor. The scFab is coupled to the C-terminal end of the CH2-CH3 Ig domain by a linker. In certain embodiments, the scFab is directed to the transferrin receptor.

The term “monovalent binding mode” denotes a specific binding to the BBBR where the interaction between the monovalent binding entity and the BBBR takes place through one single epitope. The monovalent binding mode prevents any dimerization/multimerization of the BBBR due to a single epitope interaction point. The monovalent binding mode prevents that the intracellular sorting of the BBBR is altered.

A “naked antibody” refers to an antibody that is not conjugated to a heterologous moiety (e.g., a cytotoxic moiety) or radiolabel. The naked antibody may be present in a pharmaceutical composition.

“Native antibodies” refer to naturally occurring immunoglobulin molecules with varying structures. For example, native IgG antibodies are heterotetrameric glycoproteins of about 150,000 Daltons, composed of two identical light chains and two identical heavy chains that are disulfide-bonded. From N- to C-terminus, each heavy chain has a variable domain (VH), also called a variable heavy domain or a heavy chain variable region, followed by three constant heavy domains (CH1, CH2, and CH3). Similarly, from N- to C-terminus, each light chain has a variable domain (VL), also called a variable light domain or a light chain variable region, followed by a constant light (CL) domain.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

The term “sample” includes, but is not limited to, any quantity of a substance from a living thing or formerly living thing. Such living things include mice, monkeys, rats, rabbits, and other animals. In one embodiment, the sample is obtained from a monkey, especially a cynomolgus monkey, or a rabbit, or a mouse, or a rat.

The term “signal” as used herein encompasses any detectable physical change that can be used to indicate that a reaction has taken place, for example, binding of an antibody to its antigen. It is contemplated that signals in the form of fluorimetric or colorimetric products/reagents are specific forms of a signal and can be used in the method according to the current invention. In some embodiments of the present invention, the signal is assessed quantitatively.

The term “solid phase” denotes a non-fluid substance, and includes particles (including microparticles and beads) made from materials such as polymer, metal (paramagnetic, ferromagnetic particles), glass, and ceramic; gel substances such as silica, alumina, and polymer gels; capillaries, which may be made of polymer, metal, glass, and/or ceramic; zeolites and other porous substances; electrodes; microtiter plates; solid strips; and cuvettes, tubes or other spectrometer sample containers. A solid phase component is distinguished from inert solid surfaces in that a “solid phase” contains at least one moiety on its surface, which is intended to interact with a substance in a sample. A solid phase may be a stationary component, such as a tube, strip, cuvette or microtiter plate, or may be non-stationary components, such as beads and microparticles. A variety of microparticles that allow either non-covalent or covalent attachment of proteins and other substances may be used. Such particles include polymer particles such as polystyrene and poly (methyl methacrylate); gold particles such as gold nanoparticles and gold colloids; and ceramic particles such as silica, glass, and metal oxide particles. See for example Martin, C. R., et al., Analytical Chemistry-News & Features, 70 (1998) 322A-327A, or Butler, J. E., Methods 22 (2000) 4-23.

The terms “therapeutic (monoclonal) antibody” and “drug” are used interchangeably herein. These terms are used in the broadest sense and encompasses various antibody structures, including but not limited to monoclonal antibodies, polyclonal antibodies, and antibody fragments so long as they exhibit the desired antigen-binding activity.

The “transferrin receptor” (“TfR”) is a transmembrane glycoprotein (with a molecular weight of about 180,000 Da) composed of two disulphide-bonded sub-units (each of apparent molecular weight of about 90,000 Da) involved in iron uptake in vertebrates. In one embodiment, the TIR as mentioned herein is human TfR comprising the amino acid sequence as in Schneider et al (Nature 311 (1984) 675-678), for example.

### Multispecific Antibodies

In certain embodiments, the therapeutic antibody is a bispecific antibody. In one embodiment, the therapeutic antibody is a bispecific, trivalent antibody. In one preferred embodiment, the therapeutic antibody is a monoclonal, bispecific, trivalent antibody.

In certain embodiments, the therapeutic antibody is a multispecific antibody, e.g. a bispecific antibody. Multispecific antibodies are monoclonal antibodies that have binding specificities for at least two different antigens. In certain embodiments, one of the binding specificities is for a first antigen and the other is for a different second antigen. Bispecific antibodies can be prepared as full-length antibodies or antibody fragments. In one embodiment, the antibody is a bispecific antibody, which specifically binds to a first and a second antigen. In one embodiment, the bispecific antibody has i) a first binding specificity that specifically binds to a first antigen, and ii) a second binding specificity that specifically binds to a second antigen. In one embodiment, the antibody is a bispecific, trivalent antibody. In one preferred embodiment, the antibody is a monoclonal, bispecific, trivalent antibody.

In one embodiment, one of the binding sites specifically binds to a BBBR.

Techniques for making multispecific antibodies include, but are not limited to, recombinant co-expression of two immunoglobulin heavy chain-light chain pairs having different specificities (see Milstein, C. and Cuello, A. C., Nature 305 (1983) 537-540, WO 93/08829, and Traunecker, A., et al., EMBO J. 10 (1991) 3655-3659), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004); cross-linking two or more antibodies or fragments (see, e.g., U.S. Pat. No. 4,676,980, and Brennan, M., et al., Science 229 (1985) 81-83); using leucine zippers to produce bi-specific antibodies (see, e.g., Kostelny, S. A., et al., J. Immunol. 148 (1992) 1547-1553; using “diabody” technology for making bispecific antibody fragments (see, e.g., Holliger, P., et al., Proc. Natl. Acad. Sci. USA 90 (1993) 6444-6448); and using single-chain Fv (scFv) dimers (see, e.g., Gruber, M., et al., J. Immunol. 152 (1994) 5368-5374); and preparing trispecific antibodies as described, e.g., in Tutt, A., et al., J. Immunol. 147 (1991) 60-69).

Multispecific antibodies are described in WO 2009/080251, WO 2009/080252, WO 2009/080253, WO 2009/080254, WO 2010/112193, WO 2010/115589, WO 2010/136172, WO 2010/145792, or WO 2010/145793.

Different bispecific antibody formats are known.

Exemplary bispecific antibody formats for which the methods as reported herein can be used are
the CrossMab format (=CrossMab): a multispecific IgG antibody comprising a first Fab fragment and a second Fab fragment, wherein in the first Fab fragment
   a) only the CH1 and CL domains are replaced by each other (i.e. the light chain of the first Fab fragment comprises a VL and a CH1 domain and the heavy chain of the first Fab fragment comprises a VH and a CL domain);
   b) only the VH and VL domains are replaced by each other (i.e. the light chain of the first Fab fragment comprises a VH and a CL domain and the heavy chain of the first Fab fragment comprises a VL and a CH1 domain); or
   c) the CH1 and CL domains are replaced by each other and the VH and VL domains are replaced by each other (i.e. the light chain of the first Fab fragment comprises a VH and a CH1 domain and the heavy chain of the first Fab fragment comprises a VL and a CL domain); and
wherein the second Fab fragment comprises a light chain comprising a VL and a CL domain, and a heavy chain comprising a VH and a CH1 domain;
the CrossMab may comprises a first heavy chain including a CH3 domain and a second heavy chain including a CH3 domain, wherein both CH3 domains are engineered in a complementary manner by respective amino acid substitutions, in order to support heterodimerization of the first heavy chain and the modified second heavy chain, e.g. as disclosed in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, or WO 2013/096291 (incorporated herein by reference);
the one-armed single chain format (=one-armed single chain antibody): antibody comprising a first binding site that specifically binds to a first antigen and a second binding site that specifically binds to a second antigen, whereby the individual chains are as follows
   light chain (variable light chain domain+light chain kappa constant domain)
   combined light/heavy chain (variable light chain domain+light chain constant domain+peptidic linker+variable heavy chain domain+CH1+Hinge+CH2+CH3 with knob mutation)
   heavy chain (variable heavy chain domain+CH1+Hinge+CH2+CH3 with hole mutation);
the two-armed single chain format (=two-armed single chain antibody): antibody comprising a first binding site that specifically binds to a first antigen and a second binding site that specifically binds to a second antigen, whereby the individual chains are as follows
   combined light/heavy chain 1 (variable light chain domain+light chain constant domain+peptidic linker+variable heavy chain domain+CH1+Hinge+CH2+CH3 with hole mutation)
   combined light/heavy chain 2 (variable light chain domain+light chain constant domain+peptidic linker+variable heavy chain domain+CH1+Hinge+CH2+CH3 with knob mutation);
the common light chain bispecific format (=common light chain bispecific antibody): antibody comprising a first binding site that specifically binds to a first antigen and a second binding site that specifically binds to a second antigen, whereby the individual chains are as follows
   light chain (variable light chain domain+light chain constant domain)
   heavy chain 1 (variable heavy chain domain+CH1+Hinge+CH2+CH3 with hole mutation)
   heavy chain 2 (variable heavy chain domain+CH1+Hinge+CH2+CH3 with knob mutation);
the bispecific Fab format: Fab comprising two (non-overlapping) paratopes in a complementary pair of a VH and a VL domain, wherein the first paratope comprises (consists of) amino acid residues from CDR1 and CDR3 of the VL domain and CDR2 of the VH domain, and the second paratope comprises (consists of) residues from CDR1 and CDR3 of the VH domain and CDR2 of the VL domain; the term “non-overlapping” in this context indicates that an amino acid residue that is comprised within the first paratope of the bispecific Fab is not comprised in the second paratope, and an amino acid that is comprised within the second paratope of the bispecific Fab is not comprised in the first paratope;
the TCB format: a bispecific antibody comprising
   a first and a second Fab fragment, wherein each binding site of the first and the second Fab fragment specifically bind to a second antigen,
   a third Fab fragment, wherein the binding site of the third Fab fragment specifically binds to a first antigen, and wherein the third Fab fragment comprises a domain crossover such that the variable light chain domain (VL) and the variable heavy chain domain (VH) are replaced by each other, and
   an Fc-region comprising a first Fc-region polypeptide and a second Fc-region polypeptide,
wherein the first and the second Fab fragment each comprise a heavy chain fragment and a full-length light chain,
wherein the C-terminus of the heavy chain fragment of the first Fab fragment is fused to the N-terminus of the first Fc-region polypeptide,
wherein the C-terminus of the heavy chain fragment of the second Fab fragment is fused to the N-terminus of the variable light chain domain of the third Fab fragment and the C-terminus of the heavy chain constant domain 1 of the third Fab fragment is fused to the N-terminus of the second Fc-region polypeptide.
the brain-shuttle format (BS): a bispecific antibody comprising
   a) one (full length) antibody comprising two pairs each of a (full length) antibody light chain and a (full length) antibody heavy chain, wherein the binding sites formed by each of the pairs of the (full length) heavy chain and the (full length) light chain specifically bind to a first antigen, and
   b) one additional Fab fragment, wherein the additional Fab fragment is fused to any C-terminus of one heavy chain of the (full length) antibody, wherein the binding site of the additional Fab fragment specifically binds to a second antigen,
   wherein the additional Fab fragment specifically binding to the second antigen comprises a domain crossover such that the constant light chain domain (CL) and the constant heavy chain domain 1 (CH1) are replaced by each other, and
   wherein the first antigen is the brain target and the second antigen is human transferrin receptor.

In one embodiment, the bispecific antibody is a CrossMab.

In one embodiment, the bispecific antibody is a one-armed single chain antibody.

In one embodiment, the bispecific antibody is a two-armed single chain antibody.

In one embodiment, the bispecific antibody is a common light chain bispecific antibody.

In one embodiment, the bispecific antibody is a bispecific Fab.

In one embodiment, the bispecific antibody is a TCB.

In one embodiment, the bispecific antibody is a BS.

Multivalent, multispecific antibodies specifically bind to different targets, most likely with different affinities and complex stabilities for each target. Only a fully active multivalent, multispecific antibody can bind to all targets and shows the full biological activity in a corresponding assay.

### A. Exemplary Bispecific Antibody: Anti-Human A-Beta/Human Transferrin Receptor Antibody

In certain embodiments, the therapeutic antibody to be determined in a method according to the current invention is an antibody that binds to human A-beta and human transferrin receptor. This antibody is a bispecific antibody consisting of a full-length core antibody and a fused Fab fragment in which certain domains are crosswise exchanged. Thus, the resulting bispecific antibody is asymmetric. Therefore, the bispecific antibodies are produced using the heterodimerization technology called knobs-into-holes using a first heavy chain with the so-called knob mutations (HCknob) and a second heavy chain with the so-called hole mutations (HChole).

Exemplary antibody 0012 is composed of four polypeptides that have the amino acid sequence of SEQ ID NO: 04 to 07.

Exemplary antibody 0015 is composed of four polypeptides that have the amino acid sequence of SEQ ID NO: 08 to 11.

Exemplary antibody 0020 is composed of three polypeptides that have the amino acid sequence of SEQ ID NO: 12 to 14.

Exemplary antibody 0024 is composed of four polypeptides that have the amino acid sequence of SEQ ID NO: 15 to 18.

In one aspect, the therapeutic antibody is a bispecific antibody comprising
a) one full length antibody comprising two pairs each of a full length antibody light chain and a full length antibody heavy chain, wherein the binding sites formed by each of the pairs of the full length heavy chain and the full length light chain specifically bind to a first antigen, and
b) one additional Fab fragment, wherein the additional Fab fragment is fused to the C-terminus of one heavy chain of the full length antibody, wherein the binding site of the additional Fab fragment specifically binds to a second antigen,
wherein each of the full length antibody light chains comprises in the constant light chain domain (CL) at position 123 the amino acid residue arginine (instead of the wild-type glutamic acid residue; E123R mutation) and at position 124 the amino acid residue lysine (instead of the wild-type glutamine residue; Q124K mutation) (numbering according to Kabat),
wherein each of the full length antibody heavy chains comprises in the first constant heavy chain domain (CH1) at position 147 an glutamic acid residue (instead of the wild-type lysine residue; K147E mutation) and at position 213 an glutamic acid residue (instead of the wild-type lysine amino acid residue; K213E mutation) (numbering according to Kabat EU index),
wherein the additional Fab fragment specifically binding to the second antigen comprises a domain crossover such that the constant light chain domain (CL) and the constant heavy chain domain 1 (CH1) are replaced by each other, and
wherein the first antigen is human A-beta protein and the second antigen is human transferrin receptor.

In another embodiment, the therapeutic antibody is a bispecific antibody comprising
a) one full length antibody comprising two pairs each of a full length antibody light chain and a full length antibody heavy chain, wherein the binding sites formed by each of the pairs of the full length heavy chain and the full length light chain specifically bind to a first antigen, and
b) one additional Fab fragment, wherein the additional Fab fragment is fused to the C-terminus of one heavy chain of the full length antibody, wherein the binding site of the additional Fab fragment specifically binds to a second antigen,
wherein each of the full length antibody light chains comprises in the constant light chain domain (CL) at position 123 the amino acid residue arginine (instead of the wild-type glutamic acid residue; E123R mutation) and at position 124 the amino acid residue lysine (instead of the wild-type glutamine residue; Q124K mutation) (numbering according to Kabat),
wherein each of the full length antibody heavy chains comprises in the first constant heavy chain domain (CH1) at position 147 an glutamic acid residue (instead of the wild-type lysine residue; K147E mutation) and at position 213 an glutamic acid residue (instead of the wild-type lysine amino acid residue; K213E mutation) (numbering according to Kabat EU index),
wherein the additional Fab fragment specifically binding to the second antigen comprises a domain crossover such that the constant light chain domain (CL) and the constant heavy chain domain 1 (CH1) are replaced by each other,
wherein the first antigen is human A-beta protein and the second antigen is human transferrin receptor,
wherein the human A-beta binding site comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 19 and a light chain variable domain (VL) sequence having at least 90%, 91%, 92%, 93%, 94%, 95%, 96%, 97%, 98%, 99%, or 100% sequence identity to the amino acid sequence of SEQ ID NO: 20, and
wherein the human transferrin receptor binding site comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 21 and a light chain variable domain (VL) sequence having at least 90%, 91%, 92%, 93%, 94%, 95%, 96%, 97%, 98%, 99%, or 100% sequence identity to the amino acid sequence of SEQ ID NO: 22.

In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but a binding site comprising that sequence retains the ability to bind to its antigen. In certain embodiments, a total of 1 to 10 amino acids have been substituted, inserted and/or deleted in SEQ ID NO: 19 or 21. In certain embodiments, substitutions, insertions, or deletions occur in regions outside the HVRs (i.e., in the FRs).

In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but a binding site comprising that sequence retains the ability to bind to its antigen. In certain embodiments, a total of 1 to 10 amino acids have been substituted, inserted and/or deleted in SEQ ID NO: 20 or 22. In certain embodiments, substitutions, insertions, or deletions occur in regions outside the HVRs (i.e., in the FRs).

In one embodiment, the human A-beta binding site comprises the VH sequence as in SEQ ID NO: 19, including post-translational modifications of that sequence and the VL sequence as in SEQ ID NO: 20.

In one embodiment, the human transferrin receptor-binding site comprises the VH sequence as in SEQ ID NO: 21, including post-translational modifications of that sequence and the VL sequence as in SEQ ID NO: 22.

In one embodiment, the bispecific antibody comprises
i) a light chain that has a sequence identity to SEQ ID NO: 23 of at least 70%, at least 80%, at least 90%, or 95% or more,
ii) a heavy chain that has a sequence identity to SEQ ID NO: 24 of at least 70%, at least 80%, at least 90%, or 95% or more,
iii) a light chain that has a sequence identity to SEQ ID NO: 25 of at least 70%, at least 80%, at least 90%, or 95% or more, and
iv) a heavy chain Fab fragment that has a sequence identity to SEQ ID NO: 26 of at least 70%. at least 80%, at least 90%, or 95% or more.
wherein

SEQ ID NO: 23 has the amino acid sequence
DIVLTQSPATLSLSPGERATLSCRASQSVSSSYLAWYQQKPGQAPRLLIY
GASSRATGVPARFSGSGSGTDFTLTISSLEPEDFATYYCLQIYNMPITFG
QGTKVEIKRTVAAPSVFIFPPSDRKLKSGTASVVCLLNNFYPREAKVQWK
VDNALQSGNSQESVTEQDSKDSTYSLSSTLTLSKADYEKHKVYACEVTHQ
GLSSPVTKSFNRGEC,
SEQ ID NO: 24 has the amino acid sequence
QVELVESGGGLVQPGGSLRLSCAASGFTFSSYAMSWVRQAPGKGLEWVSA
INASGTRTYYADSVKGRFTISRDNSKNTLYLQMNSLRAEDTAVYYCARGK
GNTHKPYGYVRYFDVWGQGTLVTVSSASTKGPSVFPLAPSSKSTSGGTAA
LGCLVEDYFPEPVTVSWNSGALTSGVHTFPAVLQSSGLYSLSSVVTVPSS
SLGTQTYICNVNHKPSNTKVDEKVEPKSCDKTHTCPPCPAPELLGGPSVF
LFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKG
QPREPQVCTLPPSRDELTKNQVSLSCAVKGFYPSDIAVEWESNGQPENNY
KTTPPVLDSDGSFFLVSKLTVDKSRWQQGNVFSCSVMHEALHNHYTQKSL
SLSPG,
SEQ ID NO: 25 has the amino acid sequence
AIQLTQSPSSLSASVGDRVTITCRASQSISSYLAWYQQKPGKAPKLLIYR
ASTLASGVPSRFSGSGSGTDFTLTISSLQPEDFATYYCQQNYASSNVDNT
FGGGTKVEIKSSASTKGPSVFPLAPSSKSTSGGTAALGCLVKDYFPEPVT
VSWNSGALTSGVHTFPAVLQSSGLYSLSSVVTVPSSSLGTQTYICNVNHK
PSNTKVDKKVEPKSC,
and
SEQ ID NO: 26 has the amino acid sequence
QSMQESGPGLVKPSQTLSLTCTVSGFSLSSYAMSWIRQHPGKGLEWIGYI
WSGGSTDYASWAKSRVTISKTSTTVSLKLSSVTAADTAVYYCARRYGTSY
PDYGDASGFDPWGQGTLVTVSSASVAAPSVFIFPPSDEQLKSGTASVVCL
LNNFYPREAKVQWKVDNALQSGNSQESVTEQDSKDSTYSLSSTLTLSKAD
YEKHKVYACEVTHQGLSSPVTKSFNRGEC.

In another embodiment, the therapeutic antibody is a bispecific antibody comprising
a) one full length antibody comprising two pairs each of a full length antibody light chain and a full length antibody heavy chain, wherein the binding sites formed by each of the pairs of the full length heavy chain and the full length light chain specifically bind to a first antigen, and
b) one additional Fab fragment, wherein the additional Fab fragment is fused to the C-terminus of one heavy chain of the full length antibody, wherein the binding site of the additional Fab fragment specifically binds to a second antigen,
wherein each of the full length antibody light chains comprises in the constant light chain domain (CL) at position 123 the amino acid residue arginine (instead of the wild-type glutamic acid residue; E123R mutation) and at position 124 the amino acid residue lysine (instead of the wild-type glutamine residue; Q124K mutation) (numbering according to Kabat),
wherein each of the full length antibody heavy chains comprises in the first constant heavy chain domain (CH1) at position 147 an glutamic acid residue (instead of the wild-type lysine residue; K147E mutation) and at position 213 an glutamic acid residue (instead of the wild-type lysine amino acid residue; K213E mutation) (numbering according to Kabat EU index),
wherein the additional Fab fragment specifically binding to the second antigen comprises a domain crossover such that the constant light chain domain (CL) and the constant heavy chain domain 1 (CH1) are replaced by each other,
wherein the first antigen is human A-beta protein and the second antigen is human transferrin receptor,
wherein the human A-beta binding site comprises a heavy chain variable domain (VH) that has the amino acid sequence of SEQ ID NO: 19 and a light chain variable domain (VL) that has the amino acid sequence of SEQ ID NO: 20, and
wherein the human transferrin receptor-binding site comprises a heavy chain variable domain (VH) that has the amino acid sequence of SEQ ID NO: 21 and a light chain variable domain (VL) that has the amino acid sequence of SEQ ID NO: 22.

In another embodiment, the therapeutic antibody is a bispecific antibody comprising
a) one full length antibody comprising two pairs each of a full length antibody light chain and a full length antibody heavy chain, wherein the binding sites formed by each of the pairs of the full length heavy chain and the full length light chain specifically bind to a first antigen, wherein the full length antibody comprises an Fc-region that is formed by the Fc-region polypeptides, each comprising the CH1, CH2 and CH3 domain, of the two full length heavy chains, and
b) one additional Fab fragment, wherein the additional Fab fragment is fused to the C-terminus of one heavy chain of the full length antibody, wherein the binding site of the additional Fab fragment specifically binds to a second antigen,
wherein each of the full length antibody light chains comprises in the constant light chain domain (CL) at position 123 the amino acid residue arginine (instead of the wild-type glutamic acid residue; E123R mutation) and at position 124 the amino acid residue lysine (instead of the wild-type glutamine residue; Q124K mutation) (numbering according to Kabat),
wherein each of the full length antibody heavy chains comprises in the first constant heavy chain domain (CH1) at position 147 an glutamic acid residue (instead of the wild-type lysine residue; K147E mutation) and at position 213 an glutamic acid residue (instead of the wild-type lysine amino acid residue; K213E mutation) (numbering according to Kabat EU index),
wherein the additional Fab fragment specifically binding to the second antigen comprises a domain crossover such that the constant light chain domain (CL) and the constant heavy chain domain 1 (CH1) are replaced by each other,
wherein the first antigen is human A-beta protein and the second antigen is human transferrin receptor,
wherein the human A-beta binding site comprises a heavy chain variable domain (VH) that has the amino acid sequence of SEQ ID NO: 19 and a light chain variable domain (VL) that has the amino acid sequence of SEQ ID NO: 20,
wherein the human transferrin receptor binding site comprises a heavy chain variable domain (VH) that has the amino acid sequence of SEQ ID NO: 21 and a light chain variable domain (VL) that has the amino acid sequence of SEQ ID NO: 22, and
wherein the Fc-region polypeptides are
a) of the human subclass IgG1,
b) of the human subclass IgG4,
c) of the human subclass IgG1 with the mutations L234A, L235A and P329G,
d) of the human subclass IgG4 with the mutations S228P, L235E and P329G,
e) of the human subclass IgG1 with the mutations L234A, L235A and P329G in both Fc-region polypeptides and the mutations T366W and S354C in one Fc-region polypeptide and the mutations T366S, L368A, Y407V and Y349C in the respective other Fc-region polypeptide,
f) of the human subclass IgG4 with the mutations S228P and P329G in both Fc-region polypeptides and the mutations T366W and S354C in one Fc-region polypeptide and the mutations T366S, L368A, Y407V and Y349C in the respective other Fc-region polypeptide,
g) of the human subclass IgG1 with the mutations L234A, L235A, P329G, I253A, H310A and H435A in both Fc-region polypeptides and the mutations T366W and S354C in one Fc-region polypeptide and the mutations T366S, L368A, Y407V and Y349C in the respective other Fc-region polypeptide, or
h) of the human subclass IgG1 with the mutations L234A, L235A, P329G, M252Y, S254T and T256E in both Fc-region polypeptides and the mutations T366W and S354C in one Fc-region polypeptide and the mutations T366S, L368A, Y407V and Y349C in the respective other Fc-region polypeptide.

### B. Exemplary Anti-Transferrin Receptor Antibodies

The anti-transferrin receptor binding site of a therapeutic antibody to be determined in a method according to the current invention have an off-rate for binding to the human transferrin receptor that is within a certain range in order to ensure proper BBB shuttling. This range is defined at the one end by the off-rate of the murine anti-transferrin receptor antibody 128.1 (variable domain amino acid sequences given in SEQ ID NO: 27 and 28) determined by surface plasmon resonance for the cynomolgus transferrin receptor and at the other end by 5% of that off-rate (i.e. a 20-times slower dissociation). The off-rate for the human transferrin receptor should be between and including 0.1 1/s and 0.005 1/s.

One aspect as reported herein is an anti-transferrin receptor antibody that specifically binds to human transferrin receptor and cynomolgus transferrin receptor, which comprises
i) a humanized heavy chain variable domain derived from the heavy chain variable domain of SEQ ID NO: 29, and
ii) a humanized light chain variable domain derived from the light chain variable domain of SEQ ID NO: 30,
wherein the antibody has an off-rate for the human transferrin receptor that is equal to or less than (i.e. at most) the off-rate of the anti-transferrin receptor antibody 128.1 for the cynomolgus transferrin receptor,
whereby the off-rates are determined by surface plasmon resonance, and
whereby the anti-transferrin receptor antibody 128.1 has a heavy chain variable domain of SEQ ID NO: 27 and a light chain variable domain of SEQ ID NO: 28.

In one embodiment, the off-rate for the human transferrin receptor is between and including 0.1 1/s and 0.005 1/s.

In one embodiment, the antibody has in the light chain variable domain at position 80 a proline amino acid residue (P) (numbering according to Kabat).

In one embodiment, the antibody has in the light chain variable domain at position 91 an asparagine amino acid residue (N) (numbering according to Kabat).

In one embodiment, the antibody has in the light chain variable domain at position 93 an alanine amino acid residue (A) (numbering according to Kabat).

In one embodiment, the antibody has in the heavy chain variable domain at position 100g a serine amino acid residue (S) (numbering according to Kabat).

In one embodiment, the antibody has in the heavy chain variable domain at position 100g a glutamine amino acid residue (Q) (numbering according to Kabat).

In one embodiment, the antibody has in the heavy chain variable domain at position 65 a serine amino acid residue (S) (numbering according to Kabat).

In one embodiment, the antibody has in the heavy chain variable domain at position 105 a glutamine amino acid residue (Q) (numbering according to Kabat).

In one embodiment, the antibody the antibody has in the light chain variable domain at position 80 a proline amino acid residue (P), in the light chain variable domain at position 91 an asparagine amino acid residue (N), in the light chain variable domain at position 93 an alanine amino acid residue (A), in the heavy chain variable domain at position 100g a serine amino acid residue (S), in the heavy chain variable domain at position 65 a serine amino acid residue (S), and in the heavy chain variable domain at position 105 a glutamine amino acid residue (Q) (numbering according to Kabat).

In one embodiment, the antibody the antibody has in the light chain variable domain at position 80 a proline amino acid residue (P), in the light chain variable domain at position 91 an asparagine amino acid residue (N), in the light chain variable domain at position 93 an alanine amino acid residue (A), in the heavy chain variable domain at position 100g a glutamine amino acid residue (Q), in the heavy chain variable domain at position 65 a serine amino acid residue (S), and in the heavy chain variable domain at position 105 a glutamine amino acid residue (Q) (numbering according to Kabat).

Such anti-transferrin receptor bispecific antibodies can be used as blood-brain-barrier shuttle module to deliver a brain effector entity across the blood-brain-barrier into the brain. The blood-brain-barrier shuttle module is a monovalent binding entity that specifically binds to the human transferrin receptor. The anti-transferrin receptor bispecific antibodies when used as blood-brain-barrier shuttle module are useful, e.g., for the diagnosis or treatment of neurological disorders, such as Alzheimer's disease, Parkinson's Disease and Alzheimer's Disease with Parkinson's Disease co-morbidity.

In one embodiment, the anti-transferrin receptor binding site of the therapeutic antibody comprises the heavy chain variable domain of SEQ ID NO: 31 and the light chain variable domain of SEQ ID NO: 32 which reflect with respect to the human transferrin receptor the binding properties of the murine antibody 128.1 with respect to the cynomolgus transferrin receptor regarding the binding off-rate.

In one embodiment, the anti-transferrin receptor binding site of the therapeutic antibody specifically binds to human transferrin receptor (huTfR) and cynomolgus transferrin receptor (cyTfR) and comprises i) a humanized heavy chain variable domain derived from the heavy chain variable domain of SEQ ID NO: 29 and ii) a humanized light chain variable domain derived from the light chain variable domain of SEQ ID NO: 30, wherein the light chain variable domain has at position 80 a proline amino acid residue (P), at position 91 an asparagine amino acid residue (N) and at position 93 an alanine amino acid residue (A) (numbering according to Kabat).

In one embodiment, the anti-transferrin receptor binding site of the therapeutic antibody further has in the heavy chain variable domain at position 100g a serine amino acid residue (S) (numbering according to Kabat).

In one embodiment, the anti-transferrin receptor binding site of the therapeutic antibody further has in the heavy chain variable domain at position 65 a serine amino acid residue (S) (numbering according to Kabat).

In one embodiment, the anti-transferrin receptor binding site of the therapeutic antibody further has in the heavy chain variable domain at position 105 a glutamine amino acid residue (Q) (numbering according to Kabat).

In one embodiment, the anti-transferrin receptor binding site of the therapeutic antibody specifically binds to human transferrin receptor (huTfR) and cynomolgus transferrin receptor (cyTfR) and comprises i) a humanized heavy chain variable domain derived from the heavy chain variable domain of SEQ ID NO: 29 and ii) a humanized light chain variable domain derived from the light chain variable domain of SEQ ID NO: 30, wherein the therapeutic antibody has an off-rate in the unit 1/s for the human transferrin receptor that is equal to or less than (i.e. at most) the off-rate in the unit 1/s of the anti-transferrin receptor antibody 128.1 for the cynomolgus transferrin receptor, whereby the off-rates are determined by surface plasmon resonance, and whereby the anti-transferrin receptor antibody 128.1 has a heavy chain variable domain of SEQ ID NO: 27 and a light chain variable domain of SEQ ID NO: 28.

In one embodiment, the anti-transferrin receptor binding site of the therapeutic antibody has an off-rate in the unit 1/s for the human transferrin receptor that is i) equal to or less than (i.e. at most) the off-rate in the unit 1/s of the anti-transferrin receptor antibody 128.1 for the cynomolgus transferrin receptor and ii) equal to or more than (i.e. at least) 5% of the off-rate in the unit 1/s of the anti-transferrin receptor antibody 128.1 for the cynomolgus transferrin receptor.

In one embodiment, the anti-transferrin receptor binding site of the therapeutic antibody comprises (a) a HVR-H1 comprising the amino acid sequence of SEQ ID NO: 33; (b) a HVR-H2 comprising the amino acid sequence of SEQ ID NO: 34; (c) a HVR-H3 comprising the amino acid sequence of SEQ ID NO: 35, 36 or 37, in one preferred embodiment, SEQ ID NO: 36; (d) a HVR-L1 comprising the amino acid sequence of SEQ ID NO: 38; (e) a HVR-L2 comprising the amino acid sequence of SEQ ID NO: 39; and (f) a HVR-L3 comprising the amino acid sequence of SEQ ID NO: 40.

In any of the above embodiments, an anti-transferrin receptor-binding site is humanized. In one embodiment, an anti-transferrin receptor-binding site comprises HVRs as in any of the above embodiments, and further comprises an acceptor human framework, e.g. a human immunoglobulin framework or a human consensus framework.

In one embodiment, the antibody is a bispecific antibody comprising at least one pair of a heavy chain variable domain of SEQ ID NO: 31 and a light chain variable domain of SEQ ID NO: 32 forming a binding site for the transferrin receptor and at least one pair of a heavy chain variable domain of SEQ ID NO: 41 and a light chain variable domain of SEQ ID NO: 42 binding site for human CD20. In one embodiment, the heavy chain variable region comprises a replacement of the amino acid residue at Kabat position 11 with any amino acid but leucine. In one embodiment, the substitution comprises a replacement of the amino acid residue at Kabat position 11 with a nonpolar amino acid. In one preferred embodiment, the substitution comprises a replacement of the amino acid residue at Kabat position 11 in the heavy chain variable domain of SEQ ID NO: 41 with an amino acid residue selected from the group consisting of valine, leucine, isoleucine, serine, and phenylalanine.

In one embodiment, the antibody is a bispecific antibody comprising at least one pair of a heavy chain variable domain of SEQ ID NO: 31 and a light chain variable domain of SEQ ID NO: 32 forming a binding site for the transferrin receptor and at least one pair of a heavy chain variable domain of SEQ ID NO: 43 and a light chain variable domain of SEQ ID NO: 44 binding site for human alpha-synuclein.

In one embodiment, the antibody is a bispecific antibody comprising at least one pair of a heavy chain variable domain of SEQ ID NO: 31 and a light chain variable domain of SEQ ID NO: 32 forming a binding site for the transferrin receptor and at least one pair of a humanized heavy chain variable domain derived from SEQ ID NO: 45 and a humanized light chain variable domain derived from SEQ ID NO: 46 binding site for human alpha-synuclein.

In one embodiment, the antibody is a bispecific antibody comprising at least one pair of a heavy chain variable domain of SEQ ID NO: 31 and a light chain variable domain of SEQ ID NO: 32 forming a binding site for the transferrin receptor and at least one pair of a humanized heavy chain variable domain derived from SEQ ID NO: 47 and a humanized light chain variable domain derived from SEQ ID NO: 48 binding site for human alpha-synuclein.

In one embodiment, the antibody is a bispecific antibody comprising at least one pair of a heavy chain variable domain of SEQ ID NO: 31 and a light chain variable domain of SEQ ID NO: 32 forming a binding site for the transferrin receptor and at least one pair of a humanized heavy chain variable domain derived from SEQ ID NO: 49 and a humanized light chain variable domain derived from SEQ ID NO: 50 binding site for human alpha-synuclein.

In one embodiment, the antibody is a bispecific antibody comprising at least one pair of a heavy chain variable domain of SEQ ID NO: 31 and a light chain variable domain of SEQ ID NO: 32 forming a binding site for the transferrin receptor and at least one pair of a humanized heavy chain variable domain derived from SEQ ID NO: 51 and a humanized light chain variable domain derived from SEQ ID NO: 52 binding site for human alpha-synuclein.

In one embodiment, the antibody is a bispecific antibody comprising at least one pair of a heavy chain variable domain of SEQ ID NO: 31 and a light chain variable domain of SEQ ID NO: 32 forming a binding site for the transferrin receptor and at least one pair of a humanized heavy chain variable domain derived from SEQ ID NO: 53 and a humanized light chain variable domain derived from SEQ ID NO: 54 binding site for human alpha-synuclein.

In one embodiment, the antibody is a bispecific antibody comprising at least one pair of a heavy chain variable domain of SEQ ID NO: 31 and a light chain variable domain of SEQ ID NO: 32 forming a binding site for the transferrin receptor and a binding site for i) glucocerebrosidase that has the amino acid sequence of SEQ ID NO: 55, or ii) a functional variant of SEQ ID NO: 55 having at least 70% sequence identity, or iii) a functional variant of SEQ ID NO: 55 having one or more amino acid mutations, deletions or insertions, or iv) a truncated functional variant of SEQ ID NO: 55 having at least one amino acid residue at the N-terminus or the C-terminus or within the amino acid sequence deleted, or v) a combination of iii) and iv).

In another embodiment, the therapeutic antibody is a multispecific antibody. In one such embodiment, the multispecific antibody comprises a first antigen-binding site, which binds TfR, and a second antigen-binding site, which binds a brain antigen. In one such aspect, the brain antigen is selected from the group consisting of: beta-secretase 1 (BACE1), Abeta, epidermal growth factor receptor (EGFR), human epidermal growth factor receptor 2 (HER2), tau, apolipoprotein E (ApoE), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), glucocerebrosidase, and caspase 6. In another embodiment, the multispecific antibody binds both TfR and BACE1. In another embodiment, the multispecific antibody binds both TfR and Abeta. In another embodiment, the multispecific antibody binds both TfR and alpha synuclein. In another embodiment, the multispecific antibody binds both TfR and CD20. In another embodiment, the multispecific antibody binds both TfR and glucocerebrosidase. In another embodiment, the therapeutic compound is a neurological disorder therapeutic antibody.

In one embodiment, the effector function is reduced or eliminated by at least one modification of the Fc region. In one embodiment, the effector function or complement activation function is reduced or eliminated by deletion of all or a portion of the Fc region, or by engineering the antibody such that it does not include an Fc region or non-Fc region competent for effector function or complement activation function. In one embodiment, the at least one modification of the Fc region is selected from: a point mutation of the Fc region to impair binding to one or more Fc receptors selected from the following positions: 238, 239, 248, 249, 252, 254, 265, 268, 269, 270, 272, 278, 289, 292, 293, 294, 295, 296, 297, 298, 301, 303, 322, 324, 327, 329, 333, 30 335, 338, 340, 373, 376, 382, 388, 389, 414, 416, 419, 434, 435, 437, 438, and 439; a point mutation of the Fc region to impair binding to C1q selected from the following positions: 270, 322, 329, and 321; eliminating some or all of the Fc region, and a point mutation at position 132 of the CH1 domain. In one embodiment, the modification is a point mutation of the Fc region to impair binding to C1q selected from the following positions: 270, 322, 329, and 321. In another embodiment, the modification is elimination of some or all of the Fc region. In another embodiment, complement-triggering function is reduced or eliminated by deletion of all or a portion of the Fc region, or by engineering the antibody such that it does not include an Fc region that engages the complement pathway. In one embodiment, the antibody is selected from a Fab or a single chain antibody. In another embodiment, the non-Fc region of the antibody is modified to reduce or eliminate activation of the complement pathway by the antibody. In one embodiment, the modification is a point mutation of the CH1 region to impair binding to C3. In one embodiment, the point mutation is at position 132 (see, e.g., Vidarte et al., J. Biol. Chem. 276 (2001) 38217-38223).

In one aspect of the above embodiment, the affinity of the antibody for TfR is decreased, as measured relative to a wild-type antibody of the same isotype not having lowered affinity for TfR. In one such aspect, the antibody has a K_{D} or IC₅₀ for TfR of about 1 pM to about 100 μM.

In one embodiment, the antibody as reported herein is effector function silent. In one embodiment, the antibody has no effector function. In one embodiment, the antibody is of the human IgG1 subclass and has the mutations L234A, L235A and P329G in both heavy chains (numbering according to the EU index of Kabat).

In one embodiment, the antibody is
a) a full length antibody of the human subclass IgG1, or
b) a full length antibody of the human subclass IgG4, or
c) a full-length antibody of the human subclass IgG1 with the mutations L234A, L235A and P329G,
d) a full-length antibody of the human subclass IgG4 with the mutations S228P, L235E and optionally P329G,
e) a full length antibody of the human subclass IgG1 with the mutations L234A, L235A and P329G in both heavy chains and the mutations T366W and S354C in one heavy chain and the mutations T366S, L368A, Y407V and Y349C in the respective other heavy chain, or
f) a full-length antibody of the human subclass IgG4 with the mutations S228P and optionally P329G in both heavy chains and the mutations T366W and S354C in one heavy chain and the mutations T366S, L368A, Y407V and Y349C in the respective other heavy chain.

In one embodiment, the bispecific therapeutic antibody comprises
i) a homodimeric Fc-region of the human IgG1 subclass optionally with the mutations P329G, L234A and L235A, or
ii) a homodimeric Fc-region of the human IgG4 subclass optionally with the mutations P329G, S228P and L235E, or
   iii) a heterodimeric Fc-region whereof
   a) one Fc-region polypeptide comprises the mutation T366W, and the other Fc-region polypeptide comprises the mutations T366S, L368A and Y407V, or
   b) one Fc-region polypeptide comprises the mutations T366W and Y349C, and the other Fc-region polypeptide comprises the mutations T366S, L368A, Y407V, and S354C, or
   c) one Fc-region polypeptide comprises the mutations T366W and S354C, and the other Fc-region polypeptide comprises the mutations T366S, L368A, Y407V and Y349C,
   or
iv) a heterodimeric Fc-region of the human IgG4 subclass whereof both Fc-region polypeptides comprise the mutations P329G, L234A and L235A and
   a) one Fc-region polypeptide comprises the mutation T366W, and the other Fc-region polypeptide comprises the mutations T366S, L368A and Y407V, or
   b) one Fc-region polypeptide comprises the mutations T366W and Y349C, and the other Fc-region polypeptide comprises the mutations T366S, L368A, Y407V, and S354C, or
   c) one Fc-region polypeptide comprises the mutations T366W and S354C, and the other Fc-region polypeptide comprises the mutations T366S, L368A, Y407V and Y349C,
   or
v) a heterodimeric Fc-region of the human IgG4 subclass whereof both Fc-region polypeptides comprise the mutations P329G, S228P and L235E and
   a) one Fc-region polypeptide comprises the mutation T366W, and the other Fc-region polypeptide comprises the mutations T366S, L368A and Y407V, or
   b) one Fc-region polypeptide comprises the mutations T366W and Y349C, and the other Fc-region polypeptide comprises the mutations T366S, L368A, Y407V, and S354C, or
   c) one Fc-region polypeptide comprises the mutations T366W and S354C, and the other Fc-region polypeptide comprises the mutations T366S, L368A, Y407V and Y349C.

### Immunoassays

The principles of different immunoassays are described in the art. For example, Hage, D. S. (Anal. Chem. 71 (1999) 294R-304R). Lu, B., et al. (Analyst 121 (1996) 29R-32R) report the orientated immobilization of antibodies for the use in immunoassays. Avidin-biotin-mediated immunoassays are reported, for example, by Wilchek, M., and Bayer, E. A., in Methods Enzymol. 184 (1990) 467-469.

Monoclonal antibodies and their constant domains contain a number of reactive amino acid side chains for conjugating to a member of a binding pair, such as a polypeptide/protein, a polymer (e.g. PEG, cellulose or polystyrol), or an enzyme. Chemical reactive groups of amino acids are, for example, amino groups (lysins, alpha-amino groups), thiol groups (cystins, cysteines, and methionins), carboxylic acid groups (aspartic acids, glutamic acids), and sugar-alcoholic groups. Such methods are e.g. described by Aslam M., and Dent, A., in “Bioconjugation”, MacMillan Ref. Ltd. 1999, pages 50-100.

One of the most common reactive groups of antibodies is the aliphatic s-amine of the amino acid lysine. In general, nearly all antibodies contain abundant lysine. Lysine amines are reasonably good nucleophiles above pH 8.0 (pKa=9.18) and therefore react easily and cleanly with a variety of reagents to form stable bonds.

Amine-reactive reagents react primarily with lysins and the a-amino groups of proteins. Reactive esters, particularly N-hydroxy-succinimide (NHS) esters, are among the most commonly employed reagents for modification of amine groups. The optimum pH for reaction in an aqueous environment is pH 8.0 to 9.0. Isothiocyanates are amine-modification reagents and form thiourca bonds with proteins. They react with protein amines in aqueous solution (optimally at pH 9.0 to 9.5). Aldehydes react under mild aqueous conditions with aliphatic and aromatic amines, hydrazines, and hydrazides to form an imine intermediate (Schiff's base). A Schiff's base can be selectively reduced with mild or strong reducing agents (such as sodium borohydride or sodium cyanoborohydride) to derive a stable alkyl amine bond. Other reagents that have been used to modify amines are acid anhydrides. For example, diethylenetriaminepentaacetic anhydride (DTPA) is a bifunctional chelating agent that contains two amine-reactive anhydride groups. It can react with N-terminal and ε-amine groups of amino acids to form amide linkages. The anhydride rings open to create multivalent, metal-chelating arms able to bind tightly to metals in a coordination complex.

Another common reactive group in antibodies is the thiol residue from the sulfur-containing amino acid cystine and its reduction product cysteine (or half cystine). Cysteine contains a free thiol group, which is more nucleophilic than amines and is generally the most reactive functional group in a protein. Thiols are generally reactive at neutral pH, and therefore can be coupled to other molecules selectively in the presence of amines. Since free sulfhydryl groups are relatively reactive, proteins with these groups often exist with them in their oxidized form as disulfide groups or disulfide bonds. In such proteins, reduction of the disulfide bonds with a reagent such as dithiotreitol (DTT) is required to generate the reactive free thiol. Thiol-reactive reagents are those that will couple to thiol groups on polypeptides, forming thioether-coupled products. These reagents react rapidly at slight acidic to neutral pH and therefore can be reacted selectively in the presence of amine groups. The literature reports the use of several thiolating crosslinking reagents such as Traut's reagent (2-iminothiolane), succinimidyl (acetylthio) acetate (SATA), and sulfosuccinimidyl 6-[3-(2-pyridyldithio) propionamido] hexanoate (Sulfo-LC-SPDP) to provide efficient ways of introducing multiple sulfhydryl groups via reactive amino groups. Haloacetyl derivatives, e.g. iodoacetamides, form thioether bonds and are reagents for thiol modification. Further useful reagents are maleimides. The reaction of maleimides with thiol-reactive reagents is essentially the same as with iodoacetamides. Maleimides react rapidly at slight acidic to neutral pH.

Another common reactive group in antibodies are carboxylic acids. Antibodies contain carboxylic acid groups at the C-terminal position and within the side chains of aspartic acid and glutamic acid. The relatively low reactivity of carboxylic acids in water usually makes it difficult to use these groups to selectively modify polypeptides and antibodies. When this is done, the carboxylic acid group is usually converted to a reactive ester by the use of a water-soluble carbodiimide and reacted with a nucleophilic reagent such as an amine, hydrazide, or hydrazine. The amine-containing reagent should be weakly basic in order to react selectively with the activated carboxylic acid in the presence of the more highly basic ε-amines of lysine to form a stable amide bond. Protein crosslinking can occur when the pH is raised above 8.0.

Sodium periodate can be used to oxidize the alcohol part of a sugar within a carbohydrate moiety attached to an antibody to an aldehyde. Each aldehyde group can be reacted with an amine, hydrazide, or hydrazine as described for carboxylic acids. Since the carbohydrate moiety is predominantly found on the crystallizable fragment region (Fc-region) of an antibody, conjugation can be achieved through site-directed modification of the carbohydrate away from the antigen-binding site. A Schiff's base intermediate is formed, which can be reduced to an alkyl amine through the reduction of the intermediate with sodium cyanoborohydride (mild and selective) or sodium borohydride (strong) water-soluble reducing agents.

The conjugation of a tracer and/or capture and/or detection antibody to its conjugation partner can be performed by different methods, such as chemical binding, or binding via a binding pair. The term “conjugation partner” as used herein denotes e.g. solid supports, polypeptides, detectable labels, members of specific binding pairs. In one embodiment, the conjugation of the capture and/or tracer and/or detection antibody to its conjugation partner is performed by chemically binding via N-terminal and/or ε-amino groups (lysine), ε-amino groups of different lysins, carboxy-, sulfhydryl-, hydroxyl-, and/or phenolic functional groups of the amino acid backbone of the antibody, and/or sugar alcohol groups of the carbohydrate structure of the antibody. In one embodiment, the capture antibody is conjugated to its conjugation partner via a binding pair. In one preferred embodiment, the capture antibody is conjugated to biotin and immobilization to a solid support is performed via solid support immobilized avidin or streptavidin. In one embodiment, the capture antibody is conjugated to its conjugation partner via a binding pair. In one preferred embodiment, the tracer antibody is conjugated to digoxygenin by a covalent bond as detectable label.

Chromogens (fluorescent or luminescent groups and dyes), enzymes, NMR-active groups or metal particles, haptens, e.g. digoxygenin, are examples of “detectable labels”. The detectable label can also be a photoactivatable crosslinking group, e.g. an azido or an azirine group. Metal chelates, which can be detected by electrochemiluminescense, are also preferred signal-emitting groups, with particular preference being given to ruthenium chelates, e.g. a ruthenium (bispyridyl)32+chelate. Suitable ruthenium labeling groups are described, for example, in EP 0 580 979, WO 90/05301, WO 90/11511, and WO 92/14138. For direct detection, the labeling group can be selected from any known detectable marker groups, such as dyes, luminescent labeling groups such as chemiluminescent groups, e.g. acridinium esters or dioxetanes, or fluorescent dyes, e.g. fluorescein, coumarin, rhodamine, oxazine, resorufin, cyanine and derivatives thereof. Other examples of labeling groups are luminescent metal complexes, such as ruthenium or europium complexes, enzymes, e.g. as used for ELISA or for CEDIA (Cloned Enzyme Donor Immunoassay, e.g. EP-A-0 061 888), and radioisotopes.

Indirect detection systems comprise, for example, that the detection reagent, e.g., the detection antibody is labeled with a first partner of a binding pair. Examples of suitable binding pairs are antigen/antibody, biotin or biotin analogues such as aminobiotin, iminobiotin or desthiobiotin/avidin or Streptavidin, sugar/lectin, nucleic acid or nucleic acid analogue/complementary nucleic acid, and receptor/ligand, e.g., steroid hormone receptor/steroid hormone. In one preferred embodiment, the first binding pair members comprise hapten, antigen and hormone. In one preferred embodiment, the hapten is selected from the group consisting of digoxin, digoxygenin and biotin and analogues thereof. The second partner of such binding pair, e.g. an antibody, Streptavidin, etc., usually is labeled to allow for direct detection, e.g., by the labels as mentioned above.

Immunoassays can be performed generally in three different formats. One is with direct detection, one with indirect detection, or by a sandwich assay. The direct detection immunoassay uses a detection (or tracer) antibody that can be measured directly. An enzyme or other molecule allows for the generation of a signal that will produce a color, fluorescence, or luminescence that allow for the signal to be visualized or measured (radioisotopes can also be used, although it is not commonly used today). In an indirect assay a primary antibody that binds to the analyte is used to provide a defined target for a secondary antibody (tracer antibody) that specifically binds to the target provided by the primary antibody (referred to as detector or tracer antibody). The secondary antibody generates the measurable signal. The sandwich assay makes use of two antibodies, a capture and a tracer (detector) antibody. The capture antibody is used to bind (immobilize) analyte from solution or bind to it in solution. This allows the analyte to be specifically removed from the sample. The tracer (detector) antibody is used in a second step to generate a signal (either directly or indirectly as described above). The sandwich format requires two antibodies each with a distinct epitope on the target molecule. In addition, they must not interfere with one another, as both antibodies must be bound to the target at the same time.

Different principles for the determination of bispecific antibodies in an immunoassay are known to a person skilled in the art:
1) capture using
   one of the antigens;
   an anti-idiotypic antibody against one of the binding sites;
2) detection using
   the respective other antigen;
   an anti-idiotypic antibody against the respective other binding site;

These can be combined independently of each other.

### Blood-Brain-Barrier Penetrating Antibodies of the Method According to the Invention

The present invention relates in one aspect to the determination of the concentration of a bispecific antibody for use in the treatment of a disease in a patient in brain tissue,
wherein the bispecific therapeutic antibody comprises
   i) an (effector function competent) Fc-region,
   ii) two binding sites specifically binding to a first (cell surface) target, and
   iii) one binding site specifically binding to a second (cell surface) target,
wherein the treatment has reduced side effect after administration,
wherein the side effect is one or more selected from the group consisting of vasodilation, bronchoconstriction, laryngeal edema, drop of cardiac pressure, and hypothermia.

In one embodiment, the two binding sites specifically binding to the first target and the binding site specifically binding to the second target are arranged in opposite directions, i.e. one is conjugated to the N-terminus of the Fc-region and the other is conjugated to the C-terminus of the Fc-region.

In one embodiment, the first (cell surface) target and the second (cell surface) target are different.

In one embodiment, the binding sites specifically binding to the first (cell surface) target and the binding site specifically binding to the second (cell surface) target are located at opposite ends (i.e. those specifically binding to the first target are both/each at an N-terminal end of a (full length) antibody heavy chain and that to the second target is at the C-terminal end of one of the (full length) antibody heavy chains of the bispecific antibody.

In one embodiment, the binding sites specifically binding to the first (cell surface) target and the binding site specifically binding to the second (cell surface) target are located at opposite ends of the bispecific antibody, i.e. one of the binding sites specifically binding to the first target is conjugated to the first N-terminus of the Fc-region and the other is conjugated to the second N-terminus of the Fc-region and the binding site that specifically binds to the second target is conjugated to one of the C-termini of the Fc-region.

In one embodiment, the binding site specifically binding to the second (cell surface) target is linked to one of the binding sites specifically binding to the first (cell surface) target by a peptidic linker. In one embodiment, the peptidic linker has the amino acid sequence of SEQ ID NO: 56 or 57.

In one embodiment, the binding site specifically binding to a second (cell surface) target is within the Fc-region, wherein at least one structural loop region of any of a CH2 domain, a CH3 domain, or a CH4 domain comprises at least one modification enabling the binding of said at least one modified loop region to the second (cell surface) target wherein the unmodified immunoglobulin constant domain does not bind to said target.

In one embodiment, the binding sites are pairs of an antibody heavy chain variable domain and an antibody light chain variable domain.

In one embodiment, the bispecific therapeutic antibody comprises
i) a pair of a first antibody light chain and a first antibody heavy chain,
ii) a pair of a second antibody light chain and a second antibody heavy chain, and
iii) an additional antibody fragment selected from the group consisting of scFv, Fab, scFab, dAb fragment, DutaFab and CrossFab,
wherein the pair of antibody chains of i) and ii) comprise the binding sites specifically binding to the first (cell surface) target and the additional antibody fragment of iii) comprises the binding site specifically binding to the second (cell surface) target.

In one embodiment, the additional antibody fragment of iii) is conjugated either directly or via a peptidic linker either to the first antibody heavy chain or to the second antibody heavy chain. In one embodiment, the additional antibody fragment of iii) is conjugated either directly or via a peptidic linker to the C-terminus of the antibody heavy chain of i) or ii). In one embodiment, the peptidic linker has the amino acid sequence of SEQ ID NO: 56 or 57. In one embodiment, the first antibody light chain and the second antibody light chain have the same amino acid sequence and the first antibody heavy chain and the second antibody heavy chain differ by mutations required for heterodimerization. In one embodiment, the mutations required for heterodimerization are the knobs-into-hole mutations. In one embodiment, the antibody heavy chain not conjugated to the additional antibody fragment of iii) does not comprise i) the C-terminal lysine residue or ii) the C-terminal glycine-lysine dipeptide.

In one embodiment, the first target is a brain target and the second target is the human transferrin receptor. In one embodiment, the first target is a brain target and the second target is the human transferrin receptor 1.

In one embodiment, the brain target is selected from the group consisting of beta-secretase 1 (BACE1), human amyloid beta (Abeta), epidermal growth factor receptor (EGFR), human epidermal growth factor receptor 2 (HER2), human Tau protein, phosphorylated human Tau protein, apolipoprotein E4 (ApoE4), human alpha-synuclein, human CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), and caspase 6. In one preferred embodiment, the brain target is selected from the group consisting of human CD20, human Tau protein, phosphorylated human Tau protein, human alpha-synuclein and human amyloid beta protein. In one preferred embodiment, the brain target is human amyloid beta protein. In one embodiment, the brain target is selected from SEQ ID NO: 58, 59, 60, 01, 61.

In one preferred embodiment, the bispecific therapeutic antibody comprises
i) a pair of a first antibody light chain and a first antibody heavy chain comprising a first light chain variable domain and a first heavy chain variable domain, which form a first binding site specifically binding to a brain target selected from the group consisting of human CD20, human Tau protein, phosphorylated human Tau protein, human alpha-synuclein and human amyloid beta protein,
ii) a pair of a second antibody light chain and a second antibody heavy chain comprising a second light chain variable domain and a second heavy chain variable domain, which form a second binding site specifically binding to the same brain target as the first binding site,
iii) an additional antibody fragment selected from the group consisting of scFv, Fab, scFab, dAb fragment, DutaFab and CrossFab, comprising a third light chain variable domain and a third heavy chain variable domain, which form a third binding site specifically binding to the human transferrin receptor (transferrin receptor 1), and
iv) a (human) effector function competent Fc-region (of the human IgG1 subclass),
wherein the additional antibody fragment of iii) is conjugated either directly or via a peptidic linker to the C-terminus of the antibody heavy chain of i) or ii).

In one embodiment, the additional antibody fragment is a Fab fragment, which specifically bind to a second antigen, and which is fused via a peptidic linker to the C-terminus of one of the heavy chains of i) or ii), wherein the constant domains CL and CH1 of the second light chain and the second heavy chain are replaced by each other, comprising a third light chain variable domain and a third heavy chain variable domain, which form a third binding site specifically binding to the human transferrin receptor (transferrin receptor 1).

In one embodiment, the binding site specifically binding to the human transferrin receptor (transferrin receptor 1) comprises (a) a HVR-H1 comprising the amino acid sequence of SEQ ID NO: 33 or 62; (b) a HVR-H2 comprising the amino acid sequence of SEQ ID NO: 34 or 63 or 35; (c) a HVR-H3 comprising the amino acid sequence of SEQ ID NO: 36, 37 or 64; (d) a HVR-L1 comprising the amino acid sequence of SEQ ID NO: 38 or 65; (c) a HVR-L2 comprising the amino acid sequence of SEQ ID NO: 39; and (f) a HVR-L3 comprising the amino acid sequence of SEQ ID NO: 66 or 40.

In one embodiment, the binding site specifically binding to the human transferrin receptor (transferrin receptor 1) comprises (a) a HVR-H1 comprising the amino acid sequence of SEQ ID NO: 33; (b) a HVR-H2 comprising the amino acid sequence of SEQ ID NO: 34; (c) a HVR-H3 comprising the amino acid sequence of SEQ ID NO: 37; (d) a HVR-L1 comprising the amino acid sequence of SEQ ID NO: 38; (c) a HVR-L2 comprising the amino acid sequence of SEQ ID NO: 39; and (f) a HVR-L3 comprising the amino acid sequence of SEQ ID NO: 40.

In one embodiment, the therapeutic antibody comprises one pair of a heavy chain variable domain of SEQ ID NO: 31 and a light chain variable domain of SEQ ID NO: 32 forming a binding site for the transferrin receptor (transferrin receptor 1) and at least one (i.e. one or two) pair of a heavy chain variable domain of SEQ ID NO: 19 and a light chain variable domain of SEQ ID NO: 20 (each) forming a binding site for human amyloid beta protein (Abeta).

In one embodiment, the therapeutic antibody comprises one pair of a heavy chain variable domain of SEQ ID NO: 31 and a light chain variable domain of SEQ ID NO: 32 forming the binding site for the human transferrin receptor (transferrin receptor 1) and two pairs of a heavy chain variable domain of SEQ ID NO: 41 and a light chain variable domain of SEQ ID NO: 42 each forming a binding site for human CD20. In one embodiment, the heavy chain variable region comprises a replacement of the amino acid residue at Kabat position 11 with any amino acid but leucine. In one embodiment, the substitution comprises a replacement of the amino acid residue at Kabat position 11 with a nonpolar amino acid. In one preferred embodiment, the substitution comprises a replacement of the amino acid residue at Kabat position 11 in the heavy chain variable domain of SEQ ID NO: 41 with an amino acid residue selected from the group consisting of valine, leucine, isoleucine, serine, and phenylalanine.

In one embodiment, the therapeutic antibody comprises one pair of a heavy chain variable domain of SEQ ID NO: 31 and a light chain variable domain of SEQ ID NO: 32 forming the binding site for the human transferrin receptor (transferrin receptor 1) and two pairs of a heavy chain variable domain of SEQ ID NO: 43 and a light chain variable domain of SEQ ID NO: 44 each forming a binding site for human alpha-synuclein.

In one embodiment, the therapeutic antibody comprises one pair of a heavy chain variable domain of SEQ ID NO: 31 and a light chain variable domain of SEQ ID NO: 32 forming the binding site for the human transferrin receptor (transferrin receptor 1) and two pairs of a humanized heavy chain variable domain derived from SEQ ID NO: 45 and a humanized light chain variable domain derived from SEQ ID NO: 46 each forming a binding site for human alpha-synuclein.

In one embodiment, the therapeutic antibody comprises one pair of a heavy chain variable domain of SEQ ID NO: 31 and a light chain variable domain of SEQ ID NO: 32 forming the binding site for the human transferrin receptor and two pairs of a humanized heavy chain variable domain derived from SEQ ID NO: 47 and a humanized light chain variable domain derived from SEQ ID NO: 48 each forming a binding site for human alpha-synuclein.

In one embodiment, the therapeutic antibody comprises one pair of a heavy chain variable domain of SEQ ID NO: 31 and a light chain variable domain of SEQ ID NO: 32 forming the binding site for the human transferrin receptor (transferrin receptor 1) and two pairs of a humanized heavy chain variable domain derived from SEQ ID NO: 49 and a humanized light chain variable domain derived from SEQ ID NO: 50 each forming a binding site for human alpha-synuclein.

In one embodiment, the therapeutic antibody comprises one pair of a heavy chain variable domain of SEQ ID NO: 31 and a light chain variable domain of SEQ ID NO: 32 forming the binding site for the human transferrin receptor (transferrin receptor 1) and two pairs of a humanized heavy chain variable domain derived from SEQ ID NO: 51 and a humanized light chain variable domain derived from SEQ ID NO: 52 each forming a binding site for human alpha-synuclein.

In one embodiment, the therapeutic antibody comprising one pair of a heavy chain variable domain of SEQ ID NO: 31 and a light chain variable domain of SEQ ID NO: 32 forming the binding site for the human transferrin receptor (transferrin receptor 1) and two pairs of a humanized heavy chain variable domain derived from SEQ ID NO: 53 and a humanized light chain variable domain derived from SEQ ID NO: 54 each forming a binding site for human alpha-synuclein.

In one embodiment, the disease is a neurological disorder. In one embodiment, the disease is selected from the group of neurological disorders consisting of neuropathy, amyloidosis, cancer, an ocular disease or disorder, viral or microbial infection, inflammation, ischemia, neurodegenerative disease, seizure, behavioral disorders, lysosomal storage disease, Lewy body disease, post poliomyelitis syndrome, Shy-Draeger syndrome, olivopontocerebellar atrophy, Parkinson's disease, multiple system atrophy, striatonigral degeneration, tauopathies, Alzheimer disease, supranuclear palsy, prion discase, bovine spongiform encephalopathy, scrapie, Creutzfeldt-Jakob syndrome, kuru, Gerstmann-Straussler-Scheinker disease, chronic wasting disease, and fatal familial insomnia, bulbar palsy, motor neuron disease, nervous system heterodegenerative disorder, Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, Unverricht-Lundborg syndrome, dementia, Pick's disease, spinocerebellar ataxia, cancer of the CNS and/or brain, including brain metastases resulting from cancer elsewhere in the body. In one embodiment, the disease is selected from the group of neurological disorders consisting of Alzheimer's disease, Parkinson's disease, cancer of the CNS and/or brain, including brain metastases resulting from cancer elsewhere in the body, and tauopathies. In one embodiment, the disease is selected from the group of neurological disorders consisting of Alzheimer's disease, Parkinson's disease and tauopathies.

In one embodiment, the therapeutic antibody comprises an effector function competent Fc-region. In one embodiment, the effector function competent Fc-region is an Fc-region that specifically binds to/can be specifically bound by human FcγR. In one embodiment, the effector function competent Fc-region can elicit ADCC.

In one embodiment, ADCC elicited (upon injection/while binding to the second (cell surface) target) by the bispecific therapeutic antibody is lower than that elicited by a bivalent bispecific antibody that has only one, i.e. exactly one, binding site that specifically bind to the first (cell surface) target and (exactly) one binding site that specifically binds to the second (cell surface) target, i.e. one of the binding sites specifically binding to the first (cell surface) target is deleted. In one embodiment, the ADCC is 10-fold or more lower.

In one embodiment, the administration is an intravenous, subcutaneous, or intramuscular administration.

In one embodiment, the first antibody heavy chain (of i)) and the second antibody heavy chain (of ii)) form a heterodimer. In one embodiment, the first antibody heavy chain and the second antibody heavy chain comprise mutations supporting the formation of a heterodimer.

In one embodiment,
a) the antibody heavy chains are full-length antibody heavy chains of the human subclass IgG1,
b) the antibody heavy chains are full-length antibody heavy chains of the human subclass IgG4,
c) one of the antibody heavy chains is a full length antibody heavy chain of the human subclass IgG1 with the mutations T366W and optionally S354C or Y349C and the other antibody heavy chain is a full length antibody heavy chain of the human subclass IgG1 with the mutations T366S, L368A, Y407V and optionally Y349C or S354C,
d) both antibody heavy chains are full length antibody heavy chains of the human subclass IgG1 with the mutations I253A, H310A and H435A and the mutations T366W and optionally S354C or Y349C in one of the antibody heavy chains and the mutations T366S, L368A, Y407V and optionally Y349C or S354C in the respective other antibody heavy chain,
e) both antibody heavy chains are full length antibody heavy chains of the human subclass IgG1 with the mutations M252Y, S254T and T256E and the mutations T366W and optionally S354C or Y349C in one of the antibody heavy chains and the mutations T366S, L368A, Y407V and optionally Y349C or S354C in the respective other antibody heavy chain, or
f) both antibody heavy chains are antibody heavy chains of the human subclass IgG1 with the mutations T307H and N434H and the mutations T366W and optionally S354C or Y349C in one of the antibody heavy chains and the mutations T366S, L368A, Y407V and optionally Y349C or S354C in the respective other antibody heavy chain.

In one embodiment,
a) the antibody heavy chains are antibody heavy chains of the human subclass IgG1,
b) the antibody heavy chains are antibody heavy chains of the human subclass IgG4,
c) one of the antibody heavy chains is an antibody heavy chain of the human subclass IgG1 with the mutations T366W and optionally S354C or Y349C and the other antibody heavy chain is an antibody heavy chain of the human subclass IgG1 with the mutations T366S, L368A, Y407V and optionally Y349C or S354C,
d) both antibody heavy chains are antibody heavy chains of the human subclass IgG1 with the mutations I253A, H310A and H435A and the mutations T366W and optionally S354C or Y349C in one of the antibody heavy chains and the mutations T366S, L368A, Y407V and optionally Y349C or S354C in the respective other antibody heavy chain,
e) both antibody heavy chains are antibody heavy chains of the human subclass IgG1 with the mutations M252Y, S254T and T256E and the mutations T366W and optionally S354C or Y349C in one of the antibody heavy chains and the mutations T366S, L368A, Y407V and optionally Y349C or S354C in the respective other antibody heavy chain, or
f) both antibody heavy chains are antibody heavy chains of the human subclass IgG1 with the mutations T307H and N434H and the mutations T366W and optionally S354C or Y349C in one of the antibody heavy chains and the mutations T366S, L368A, Y407V and optionally Y349C or S354C in the respective other antibody heavy chain,
wherein the C-terminal lysine or glycine-lysine dipeptide is present or absent.

### EMBODIMENTS OF THE METHODS ACCORDING TO THE INVENTION

The relation between CSF, blood-brain-barrier and blood has been reviewed by Katsinelos, T., et al. (Front. Immunol. 10 (2019) 1139) as follows:
IgG levels are maintained in human serum at around 10 mg/ml. The brain is isolated from serum by the blood-brain barrier (BBB), which is impermeable to large macromolecules including IgG (Neuwelt, E. A., et al., Nat. Rev. Neurosci. 12 (2011) 169-182). The brain, instead, is bathed in cerebrospinal fluid (CSF), which is produced following the filtration of blood and transport of ions across the choroid plexus. The resulting concentration of IgG in CSF is around 500- to 1,000-fold lower than in serum. At face value, this low concentration of antibody in the brain makes CNS antigens unattractive as targets for passive immunotherapy, which is normally administered to the periphery. This is compounded by a poor understanding of the mechanisms by which steady state levels of antibody are maintained. CSF flows around the brain, before exiting the CNS along spinal and cranial nerves and via drainage to the lymphatic system (Louveau, A., et al., Nature 523 (2015) 337-341; Aspelund, A., J. Exp. Med. 212 (2015) 991-999). Intrathecally administered IgG is rapidly cleared from the brain, largely through this bulk flow and with a possible contribution of selective transport out of the brain. The neonatal Fc receptor, FcRn, is expressed in abundance at the BBB (Schlachetzki, F., et al., J. Neurochem. 81 (2002) 203-206). Given FcRn's role in transcytosis of antibodies across the placenta, it has been suggested that FcRn may perform reverse transcytosis to help maintain the low IgG environment of the CNS. There is some evidence that antibody clearance from the brain is mediated in part by the antibody Fc domain (Zhang, Y. and Pardridge, W. M., J. Neuroimmunol. 114 (2001) 168-172; Cooper, P. R., et al., Brain Res. 1534 (2013) 13-21), and export of an anti-Aβ monoclonal antibody was reduced in an FcRn-deficient mouse (Deane, R., et al., J. Neurosci. 25 (2005) 11495-11503). However, the brain concentration of peripherally administered IgG was not significantly different between wild-type mice and mice lacking FcRn (Abuqayyas, L. and Balthasar, J. P., Mol. Pharma. 10 (2013) 1505-1513).

For small experimental animals, the blood is removed from the brain prior to sampling by perfusion. For example, mice can be transcardially perfused with ice-cold PBS at a rate of 2 ml/min for 8 min. and brains are subsequently harvested.

Methods to transport therapeutic antibodies across the blood brain barrier, using multispecific antibodies, for example, bispecific or trispecific antibodies, comprising one or more than one carrier molecule, and one or more than one cargo molecule, via a receptor-mediate transcytosis pathway are currently explored. For example, a transferrin receptor (TfR)-binding antibody (and variants thereof) may be used as the carrier, and when fused to a cargo molecule produces a bispecific antibody that is able to cross the blood brain barrier (see for example Zuchero, Y. J, Y., et. al., Neuron 89 (12016) 70-82; Bien-Ly, N., ct. al. J. Exp. Med. 211 (2014) 233-244; US 2018/8002433; CA 3,000,560; which are incorporated herein by reference). Alternatively, an insulin-like growth factor 1 receptor (IGF-1R)-binding antibody may be used as a carrier, and fused to the cargo molecule, to produce a bispecific antibody that crosses the blood brain barrier (see for example WO 2015/131256; WO 2015/131257; WO 2015/131258; which are incorporated herein by reference).

For a robust and correct determination of the amount in brain, lysates of a therapeutic antibody transported across the blood-brain-barrier into the brain the interference from residual blood in the sample has to be excluded. As outlined above the resulting concentration of IgG in CSF is around 500- to 1,000-fold lower than in serum and the brain is spanned by an interwoven net of blood vessels. Thus, the chance of residual blood in brain tissue sample is not neglectable. Furthermore, even minor amounts of residual blood can severely interfere with the quantitative determination of antibody in brain tissue.

Thus, a correction, i.e. reduction, with the amount of therapeutic antibody in residual blood in the brain lysate sample has to be made.

Thus, the use of a quantitative blood correction marker that does not significantly diffuse during the perfusion phase into the brain is required. However, if these are determined at steady state, a small, constant concentration will exist behind the BBB.

The current invention is based, at least in part, on the finding that the amount of residual blood in a brain lysate can be determined by applying a correction antibody shortly before the brain sample is taken. It has been found that it is especially advantageous to use as reference antibody an antibody that is not specifically binding to any target in the experimental animal from which the brain sample is obtained, most preferably a human germline antibody.

Thus, herein is reported a method for the determination of the amount of a therapeutic antibody, which has been transported across the blood-brain-barrier from the blood into the brain of an experimental animal. The amount is preferably determined in a brain lysate sample. The gist of the invention lies in the additional application of an inert antibody, which is not transported across the blood-brain-barrier, shortly before obtaining the brain sample in which the amount of the therapeutic antibody transported across the blood-brain-barrier has to be determined. By applying the inert antibody, a correction value for the amount of therapeutic antibody present in residual blood in the brain sample is obtained. This residual blood-derived amount is used to correct the determined amount for non-brain-located antibody. A determination without correction would determine the total amount of therapeutic antibody in the sample, i.e. the amount transported across the blood-brain-barrier into the brain and the amount in residual blood in the sample. The amount of therapeutic antibody in residual blood is not neglectable, as only about 0.1% of the antibody in the blood will pass the blood-brain-barrier. Thus, the concentration of the therapeutic antibody in the blood exceeds the concentration of the therapeutic antibody in the brain by at least two and up to three orders of magnitude. Thereby the results obtained are too high if not corrected with a method according to the current invention.

This is especially important for the comparator IgG or brain shuttles with clearance approaching that of an IgG, because slowly clearing molecules maintain a high concentration in the blood, and if the amount is comparatively very small in the brain, then a small amount of blood contamination can overwhelm the determination of brain concentrations.

The method according to the current invention can be applied to any brain tissue sample independently of the method used for removing blood therefrom.

The method according to the invention has a cross-species assay availability, sufficient assay-robustness, -precision and -accuracy and a broad sensitivity range.

In a nutshell, the current invention provides a method for determining residual blood in a brain sample of an experimental animal,
wherein just prior to perfusion, i.e. at most 5 min. before, a second inert IgG is administered, a plasma sample is taken, perfusion is conducted, brain and plasma concentrations are measured and corrected
wherein the advantage is that the amount that transits the blood-brain-barrier is limited; thereby, the concentration of the second inert antibody reflects only plasma volume
wherein a first specific assay for the therapeutic antibody and a second specific assay for the second inert antibody is employed
wherein issues with anti-therapeutic antibody antibody positive animals, which may confound the measurement, could be prevented.

FIG. 1 provides an exemplary calculation exploiting the use of an inter antibody to determine blood contamination in a (brain) tissue sample.

For example, using a conventional ELISA the concentration of the therapeutic monoclonal antibody (tmAb) and the inert reference monoclonal antibody (refmAb) is determined in the blood plasma as well as in the homogenized brain tissue sample. The result of an ELISA is normally obtained as a mass concentration with the SI unit [g/L]. In a first step each of the mass concentrations determined for the tmAb and the refmAb, respectively, is converted into a mass fraction with the unit [g/g] by dividing the determined mass concentration by the brain tissue concentration of the sample. In the second step the amount of residual plasma in the brain tissue sample, i.e. the plasma-contamination, is calculated by dividing the mass fraction of the inert antibody obtained in the first step by the determined plasma concentration of the refmAb. Thereby the volume of residual plasma per weight of brain sample is obtained. In a third step the mass fraction of tmAb in the brain tissue sample originating from plasma contamination is calculated by multiplying the plasma concentration of the tmAb with the volume of residual plasma per weight of brain sample. In the fourth and final step, the true brain concentration of the tmAb is obtained by subtracting the mass fraction of tmAb in the brain tissue sample originating from plasma contamination obtained in the third step from the mass fraction determined for the tmAb in the first step.

The method according to the invention has been applied to the analysis of two bispecific antibodies, binding to TfR and a therapeutic target 1 or 2, respectively, in cynomolgus monkey brain lysates. The respective structure of the antibodies is shown in FIG. 2. The detection assays for the therapeutic antibody and the reference antibody, respectively, are shown in FIG. 3.

As outlined in Example 1 the assay for the determination of the inert antibody has sensitivity of 8 ng/ml, i.e. about 1.1 -1.5 μL blood plasma/g cynomolgus brain can be detected (this corresponds to about 2.2-3 μL blood/g cynomolgus brain).

Five different brain regions have been analyzed: Cerebellum, Hippocampus, Statium, Cortex and Choroid plexus.

Four different animals have been analyzed, whereof animal 1 to 3 contained no residual blood in the brain samples but animal 4 did as determined by photo analysis (data not shown).

With the method according to the current invention, this contamination could be detected and, thus, the respective values could be corrected accordingly.

| | | | DP47GS | | | DP47GS |
|---|---|---|---|---|---|---|
| | | | PGLALA | plasma | blood | PGLALA |
| | | | [ng/mL] | concentration | contamination | [μg/mL] |
| animal | time | Sample tissue | MW | [μL plasma/g brain] | [μL blood/g brain] | in CPP |
| 1 | 336 h | Cerebellum | BLQ | | | 25 |
| | | Hippocampus | BLQ | | | |
| | | Striatum | BLQ | | | |
| | | Cortex | BLQ | | | |
| | | Choroid plexus | BLQ | | | |
| 2 | 336 h | Cerebellum | BLQ | | | 23 |
| | | Hippocampus | BLQ | | | |
| | | Striatum | BLQ | | | |
| | | Cortex | BLQ | | | |
| | | Choroid plexus | BLQ | | | |
| 3 | 336 h | Cerebellum | BLQ | | | 33 |
| | | Hippocampus | BLQ | | | |
| | | Striatum | BLQ | | | |
| | | Cortex | BLQ | | | |
| | | Choroid plexus | BLQ | | | |
| 4 | 336 h | Cerebellum | 18 | 1.6 | 3 | 27 |
| | | Hippocampus | BLQ | | | |
| | | Striatum | 9 | 1.3 | 3 | |
| | | Cortex | 17 | 2.1 | 4 | |
| | | Choroid plexus | BLQ | | | |

| | determined concentration | | | | | | | |
|---|---|---|---|---|---|---|---|---|
| | | plasma | | brain [ng/g] | | | | Choroid |
| animal | | [ng/mL] | [ng/μL] | Cerebellum | Hippocampus | Striatum | Cortex | plexus |
| 1 | antibody_1 | 35 | 0.034741 | 10.9 | 50.5 | 21.0 | 13.4 | 334 |
| 2 | antibody_1 | 184 | 0.184316 | 21.5 | 47.5 | 28.7 | 21.8 | 744 |
| 3 | antibody_1 | 20 | 0.019709 | 17.3 | 46.1 | 24.4 | 24.6 | 747 |
| 4 | antibody_1 | 101 | 0.101429 | 17.5 | 45.1 | 28.2 | 25.1 | 385 |

| | | | | | |
|---|---|---|---|---|---|
| brain tissue concentration | 0.422 | 0.164 | 0.262 | 0.295 | 0.019 |
| [g/mL]-> | | | | | |
| corrected brain concentration | 17.3 | | 27.9 | 24.9 | |
| [ng/g]-> | | | | | |

| | | plasma | | brain [ng/mL] | | | | Choroid |
|---|---|---|---|---|---|---|---|---|
| | | [ng/mL] | [ng/μL] | Cerebellum | Hippocampus | Striatum | Cortex | plexus |
| 1 | inert | 25107 | 25.107 | BLQ | BLQ | BLQ | BLQ | BLQ |
| | reference | | | | | | | |
| | antibody | | | | | | | |
| 2 | inert | 25807 | 25.807 | BLQ | BLQ | BLQ | BLQ | BLQ |
| | reference | | | | | | | |
| | antibody | | | | | | | |
| 3 | inert | 33193 | 33.193 | BLQ | BLQ | BLQ | BLQ | BLQ |
| | reference | | | | | | | |
| | antibody | | | | | | | |
| 4 | inert | 27212 | 27.212 | 18 | BLQ | 9 | 17 | BLQ |
| | reference | | | | | | | |
| | antibody | | | | | | | |

In a further study fifteen animals have been does with an anti-Abeta antibody at 20 mg/kg and fifteen animals have been does with 10 mg/kg of an anti-Abeta/TfR antibody. After different time points after administration, the respective samples have been analyzed. In all sample residual blood has been detected in the respective brain tissue samples. Thus, also in these cases corrected values were obtained with the method according to the current invention.

| | | | | | antibody | plasma |
|---|---|---|---|---|---|---|
| | time after | | | | ng/g | μl/g |
| analyte | application | | animal | Sample tissue | tissue | tissue |
| Anti-Abeta | 4 | h | 29211 | Brain (Cerebellum) | 22 | 1.0 |
| antibody | | | | Brain (Hippocampus) | 21 | 0.9 |
| | | | | Brain (Striatum) | 20 | 0.9 |
| | | | | Brain (Cortex) | 26 | 1.2 |
| Anti-Abeta | 4 | h | 29213 | Brain (Cerebellum) | 16 | 0.6 |
| antibody | | | | Brain (Hippocampus) | 20 | 0.7 |
| | | | | Brain (Striatum) | 13 | 0.5 |
| | | | | Brain (Cortex) | 15 | 0.6 |
| | | | | Brain (Choroid plexus) | 822 | 29.6 |
| Anti-Abeta | 4 | h | 29214 | Brain (Cerebellum) | 31 | 1.2 |
| antibody | | | | Brain (Hippocampus) | 38 | 1.4 |
| | | | | Brain (Striatum) | 21 | 0.8 |
| | | | | Brain (Cortex) | 25 | 1.0 |
| | | | | Brain (Choroid plexus) | 569 | 21.7 |
| Anti-Abeta | 24 | h | 29328 | Brain (Cerebellum) | 53 | 3.7 |
| antibody | | | | Brain (Hippocampus) | 31 | 2.1 |
| | | | | Brain (Striatum) | 22 | 1.5 |
| | | | | Brain (Cortex) | 13 | 0.9 |
| | | | | Brain (Choroid plexus) | 925 | 63.8 |
| Anti-Abeta | 24 | h | 29329 | Brain (Cerebellum) | 22 | 1.1 |
| antibody | | | | Brain (Hippocampus) | 22 | 1.0 |
| | | | | Brain (Striatum) | 21 | 1.0 |
| | | | | Brain (Cortex) | 23 | 1.1 |
| | | | | Brain (Choroid plexus) | 925 | 43.6 |
| Anti-Abeta | 24 | h | 29330 | Brain (Cerebellum) | 18 | 0.9 |
| antibody | | | | Brain (Hippocampus) | 25 | 1.3 |
| | | | | Brain (Striatum) | 25 | 1.2 |
| | | | | Brain (Cortex) | 28 | 1.4 |
| | | | | Brain (Choroid plexus) | 493 | 25.0 |
| Anti-Abeta | 96 | h | 29208 | Brain (Cerebellum) | 29 | 1.1 |
| antibody | | | | Brain (Hippocampus) | 32 | 1.2 |
| | | | | Brain (Striatum) | 32 | 1.2 |
| | | | | Brain (Cortex) | 35 | 1.3 |
| | | | | Brain (Choroid plexus) | 411 | 15.0 |
| Anti-Abeta | 96 | h | 29217 | Brain (Cerebellum) | 19 | 0.6 |
| antibody | | | | Brain (Hippocampus) | 42 | 1.3 |
| | | | | Brain (Striatum) | 22 | 0.7 |
| | | | | Brain (Cortex) | 23 | 0.7 |
| | | | | Brain (Choroid plexus) | 925 | 28.8 |
| Anti-Abeta | 96 | h | 29219 | Brain (Cerebellum) | 21 | 0.6 |
| antibody | | | | Brain (Hippocampus) | 21 | 0.6 |
| | | | | Brain (Striatum) | 16 | 0.5 |
| | | | | Brain (Cortex) | 28 | 0.8 |
| | | | | Brain (Choroid plexus) | 529 | 15.7 |
| Anti-Abeta | 168 | h | 29114 | Brain (Cerebellum) | 33 | 1.1 |
| antibody | | | | Brain (Hippocampus) | 23 | 0.7 |
| | | | | Brain (Striatum) | 32 | 1.0 |
| | | | | Brain (Cortex) | 22 | 0.7 |
| | | | | Brain (Choroid plexus) | 493 | 15.9 |
| Anti-Abeta | 168 | h | 29119 | Brain (Cerebellum) | 20 | 1.1 |
| antibody | | | | Brain (Hippocampus) | 19 | 1.0 |
| | | | | Brain (Striatum) | 26 | 1.4 |
| | | | | Brain (Cortex) | 28 | 1.5 |
| | | | | Brain (Choroid plexus) | 569 | 30.7 |
| Anti-Abeta | 168 | h | 29122 | Brain (Cerebellum) | 24 | 1.1 |
| antibody | | | | Brain (Hippocampus) | 18 | 0.8 |
| | | | | Brain (Striatum) | 26 | 1.2 |
| | | | | Brain (Cortex) | 21 | 1.0 |
| | | | | Brain (Choroid plexus) | 925 | 42.3 |
| Anti-Abeta | 336 | h | 29140 | Brain (Cerebellum) | 34 | 1.5 |
| antibody | | | | Brain (Hippocampus) | 20 | 0.9 |
| | | | | Brain (Striatum) | 37 | 1.7 |
| | | | | Brain (Cortex) | 54 | 2.5 |
| | | | | Brain (Choroid plexus) | 569 | 25.7 |
| Anti-Abeta | 336 | h | 29191 | Brain (Cerebellum) | 22 | 0.8 |
| antibody | | | | Brain (Hippocampus) | 23 | 0.9 |
| | | | | Brain (Striatum) | 21 | 0.8 |
| | | | | Brain (Cortex) | 22 | 0.8 |
| | | | | Brain (Choroid plexus) | 925 | 34.7 |
| Anti-Abeta | 336 | h | 29194 | Brain (Cerebellum) | 59 | 2.9 |
| antibody | | | | Brain (Hippocampus) | 22 | 1.1 |
| | | | | Brain (Striatum) | 16 | 0.8 |
| | | | | Brain (Cortex) | 33 | 1.6 |
| | | | | Brain (Choroid plexus) | 740 | 36.2 |
| Anti-Abeta/TfR | 4 | h | 29317 | Brain (Cerebellum) | 22 | 0.8 |
| antibody | | | | Brain (Hippocampus) | 32 | 1.2 |
| | | | | Brain (Striatum) | 20 | 0.8 |
| | | | | Brain (Cortex) | 23 | 0.9 |
| | | | | Brain (Choroid plexus) | 206 | 7.9 |
| Anti-Abeta/TfR | 4 | h | 29319 | Brain (Cerebellum) | 13 | 0.6 |
| antibody | | | | Brain (Hippocampus) | 23 | 1.1 |
| | | | | Brain (Striatum) | 28 | 1.3 |
| | | | | Brain (Cortex) | 13 | 0.6 |
| | | | | Brain (Choroid plexus) | 673 | 30.8 |
| Anti-Abeta/TfR | 4 | h | 29331 | Brain (Cerebellum) | 44 | 1.6 |
| antibody | | | | Brain (Hippocampus) | 22 | 0.8 |
| | | | | Brain (Striatum) | 14 | 0.5 |
| | | | | Brain (Cortex) | 19 | 0.7 |
| | | | | Brain (Choroid plexus) | 617 | 21.6 |
| Anti-Abeta/TfR | 24 | h | 29197 | Brain (Cerebellum) | 32 | 1.2 |
| antibody | | | | Brain (Hippocampus) | 23 | 0.9 |
| | | | | Brain (Striatum) | 69 | 2.7 |
| | | | | Brain (Cortex) | 22 | 0.8 |
| | | | | Brain (Choroid plexus) | 308 | 11.9 |
| Anti-Abeta/TfR | 24 | h | 29199 | Brain (Cerebellum) | 21 | 0.7 |
| antibody | | | | Brain (Hippocampus) | 20 | 0.6 |
| | | | | Brain (Striatum) | 16 | 0.5 |
| | | | | Brain (Cortex) | 19 | 0.6 |
| | | | | Brain (Choroid plexus) | 296 | 9.6 |
| Anti-Abeta/TfR | 24 | h | 29201 | Brain (Cerebellum) | 21 | 0.6 |
| antibody | | | | Brain (Hippocampus) | 19 | 0.6 |
| | | | | Brain (Striatum) | 18 | 0.6 |
| | | | | Brain (Cortex) | 17 | 0.5 |
| | | | | Brain (Choroid plexus) | 389 | 11.7 |
| Anti-Abeta/TfR | 96 | h | 29207 | Brain (Cerebellum) | 16 | 0.6 |
| antibody | | | | Brain (Hippocampus) | 21 | 0.8 |
| | | | | Brain (Striatum) | 25 | 0.9 |
| | | | | Brain (Cortex) | 30 | 1.1 |
| | | | | Brain (Choroid plexus) | 389 | 14.3 |
| Anti-Abeta/TfR | 96 | h | 29218 | Brain (Cerebellum) | 26 | 1.0 |
| antibody | | | | Brain (Hippocampus) | 24 | 0.9 |
| | | | | Brain (Striatum) | 16 | 0.6 |
| | | | | Brain (Cortex) | 23 | 0.9 |
| | | | | Brain (Choroid plexus) | 822 | 32.1 |
| Anti-Abeta/TfR | 96 | h | 29220 | Brain (Cerebellum) | 17 | 0.7 |
| antibody | | | | Brain (Hippocampus) | 15 | 0.6 |
| | | | | Brain (Striatum) | 21 | 0.9 |
| | | | | Brain (Cortex) | 17 | 0.7 |
| | | | | Brain (Choroid plexus) | 389 | 16.3 |
| Anti-Abeta/TfR | 168 | h | 29118 | Brain (Cerebellum) | 17 | 0.6 |
| | | | | Brain (Hippocampus) | 19 | 0.7 |
| antibody | | | | Brain (Striatum) | 19 | 0.7 |
| | | | | Brain (Cortex) | 20 | 0.7 |
| | | | | Brain (Choroid plexus) | 493 | 18.4 |
| Anti-Abeta/TfR | 168 | h | 29141 | Brain (Cerebellum) | 21 | 1.0 |
| antibody | | | | Brain (Hippocampus) | 32 | 1.6 |
| | | | | Brain (Striatum) | 45 | 2.2 |
| | | | | Brain (Cortex) | 20 | 1.0 |
| | | | | Brain (Choroid plexus) | 1057 | 51.9 |
| Anti-Abeta/TfR | 168 | h | 29157 | Brain (Cerebellum) | 24 | 0.7 |
| antibody | | | | Brain (Hippocampus) | 24 | 0.6 |
| | | | | Brain (Striatum) | 50 | 1.4 |
| | | | | Brain (Cortex) | 18 | 0.5 |
| | | | | Brain (Choroid plexus) | 1057 | 28.8 |
| Anti-Abeta/TfR | 240 | h | 28640 | Brain (Cerebellum) | 29 | 1.4 |
| antibody | | | | Brain (Hippocampus) | 22 | 1.0 |
| | | | | Brain (Striatum) | 20 | 0.9 |
| | | | | Brain (Cortex) | 19 | 0.9 |
| | | | | Brain (Choroid plexus) | 1057 | 49.9 |
| Anti-Abeta/TfR | 240 | h | 28641 | Brain (Cerebellum) | 14 | 0.4 |
| antibody | | | | Brain (Hippocampus) | 16 | 0.5 |
| | | | | Brain (Striatum) | 26 | 0.8 |
| | | | | Brain (Cortex) | 19 | 0.6 |
| | | | | Brain (Choroid plexus) | 463 | 14.5 |
| Anti-Abeta/TfR | 240 | h | 29139 | Brain (Cerebellum) | 21 | 0.9 |
| antibody | | | | Brain (Hippocampus) | 17 | 0.7 |
| | | | | Brain (Striatum) | 22 | 0.9 |
| | | | | Brain (Cortex) | 12 | 0.5 |
| | | | | Brain (Choroid plexus) | 218 | 9.4 |

To show the general applicability of the method according to the current invention the same analysis has been done with a second antibody, an anti-TfR/target_2 antibody, in C57BL/6 wild-type mice.

An overlay of the calibration curves of the detection assay of the inert reference antibody in the presence of 1% cynomolgus brain lysate (CBL; cynoBL) and 1% mouse brain lysate (MBL; muBL) is shown in FIG. 4. It can be seen that the origin of the matrix does not influence the assay.

The working range for the assay in the presence of 1% MBL is 8.4 ng/mL to 250 ng/mL. Up to 10 μg/mL of therapeutic antibody can be present in the assay without interference in the presence of 1% MBL.

The working range for the assay in the presence of 1% mouse pooled plasma (MPP) is 11 ng/ml to 220 ng/ml. Up to 20 μg/mL of therapeutic antibody can be present in the assay without interference in the presence of 1% MPP.

A single dose of 20 mg/ml of the antibody was applied and sample were analyzed 24 h, 48 h, 96 h, 168 h, 336 h, 504 h and 672 h after application. The respective concentrations in brain lysate and plasma have been determined. In FIG. 5, the determined concentrations of the applied antibody in brain lysate are shown as ratio of uncorrected vs. corrected brain concentration. That is, if the correction does not influence the value then the ratio is 1. If due to the correction by the residual plasma value the determined concentration of the second antibody has been reduced the value will become smaller than 1. This difference increases by time as more antibody is transported across the blood-brain-barrier. It can be seen from FIG. 5 that the ratio becomes smaller and smaller by time. Thereby it can be seen that the correction done according to the method according to the current invention eliminated interference from residual blood in the brain samples. The respective assay used for the determination of the second antibody is shown in FIG. 6.

### Inert Reference Monoclonal Antibody of the Method According to the Invention

An inert reference monoclonal antibody useful in the method according to the current invention is preferably a human immunoglobulin molecule, especially a human immunoglobulin molecule that is not capable of specific binding to an antigen.

An exemplary inert reference monoclonal antibody is the antibody DP47GS. DP47GS comprises a heavy chain variable region sequence based on the human VH3-23 germline sequence and a light chain variable region sequence based on the human Vk3-20 germline sequence.

In one embodiment, said inert reference monoclonal antibody is an IgG-class immunoglobulin molecule, particularly an IgG1-subclass immunoglobulin molecule. In one embodiment, said inert reference monoclonal antibody is a human immunoglobulin molecule. In one embodiment, said inert reference monoclonal antibody is a monoclonal antibody. In one embodiment, said inert reference monoclonal antibody is not capable of specific binding to an antigen. In one embodiment, said inert reference monoclonal antibody comprises a heavy chain variable region sequence based on the human VH3-23 germline sequence. In a specific embodiment, said inert reference monoclonal antibody comprises the heavy chain variable region sequence of SEQ ID NO: 67. In one embodiment, said inert reference monoclonal antibody comprises a light chain variable region sequence based on the human Vk3-20 germline sequence. In a specific embodiment, said inert reference monoclonal antibody comprises the light chain variable region sequence of SEQ ID NO: 68. In an even more specific embodiment, said inert reference monoclonal antibody comprises the heavy chain variable region sequence of SEQ ID NO: 67 and the light chain variable region sequence of SEQ ID NO: 68. In one embodiment, said inert reference monoclonal antibody is not capable of specific binding to an antigen, and comprises a heavy chain variable region sequence based on the human VH3-23 germline sequence and a light chain variable region sequence based on the human Vk3-20 germline sequence.

In one embodiment, said inert reference monoclonal antibody comprises a heavy chain variable region sequence based on the human VH3-23 germline sequence. In a specific embodiment, said inert reference monoclonal antibody comprises a heavy chain variable region sequence that is at least 95%, 96%, 97%, 98%, 99% or 100% identical to the sequence of SEQ ID NO: 67. In one embodiment, said inert reference monoclonal antibody comprises a light chain variable region sequence based on the human Vk3-20 germline sequence. In a specific embodiment, said inert reference monoclonal antibody comprises a light chain variable region sequence that is at least 95%, 96%, 97%, 98%, 99% or 100% identical to the sequence of SEQ ID NO: 68. In an even more specific embodiment, said inert reference monoclonal antibody comprises the heavy chain variable region sequence of SEQ ID NO: 67 and the light chain variable region sequence of SEQ ID NO: 68. Immunoglobulin molecules comprising these variable region sequences are not capable of specific binding to an antigen, particularly a human antigen. They lack binding to normal tissues as well as PBMCs, have no polyreactivity and show no non-specific accumulation in vivo by imaging (data not shown). The variable region sequences are entirely based on human germline sequences, with the exception of the heavy chain CDR 3 wherein a GSG sequence has been introduced to generate a non-binding immunoglobulin.

In one embodiment, said inert reference monoclonal antibody comprises a heavy chain with a variable domain with the amino acid sequence of SEQ ID NO: 67 and a human IgG1 constant region and a light chain with a variable domain with the amino acid sequence of SEQ ID NO: 68 and a human kappa light chain constant domain. In one embodiment, said inert reference monoclonal antibody comprises in the heavy chain Fc-region the mutations L234A, L235A and P329G (numbering according to Kabat EU index).

In one embodiment, said inert reference monoclonal antibody comprises a heavy chain with the amino acid sequence of SEQ ID NO: 69 and a light chain with the amino acid sequence of SEQ ID NO: 70.

### Comparative Methods and Results

### Comparative Technical Approach:

### Correction by Residual Blood Volume Without Perfusion

Friden et al. (J. Cerebral Blood Flow & Met 30 (2010) 150-161) collected the available information from literature on brain vascular space (cf. Table 1 of Friden et al.).

Based on the most commonly used 14C-Dextran method, brain plasma value would amount for approx. 18.1 μL/g brain tissue. When applying this correction all determined values became negative.

Thus, simply assuming that the total brain plasma value would be present in a brain tissue sample was not correct.

Thus, no absolute value could be applied but a co-determined correction factor was required.

Thus, a different correction factor was required.

As perfusion will be performed, a control for remaining blood contamination is required. This is especially important for the comparator IgG or brain shuttles with clearance approaching that of an IgG—why? Because slowly clearing molecules maintain a high concentration in the blood, and if the amount is comparatively very small in the brain, then a small amount of blood contamination can overwhelm the determination of brain concentrations.

Thus, there is the need to use a quantitative blood correction marker that does not significantly diffuse during the application as well as the perfusion phase in to the brain.

### Comparative Markers:

Different other non-antibody inert reference molecules, which were, prior to their testing, deemed to be likewise suitable as a corrective means in the method according to the current invention are other endogenous proteins with high molecular weight and high endogenous blood levels.

### Determination of Complement Factor H

Different publications indicated that in cynomolgus cerebrospinal fluid (liquor cerebrospinalis) as well as cynomolgus brain lysates no complement factor H is present, wherein it is common in non-CSF or non-brain tissue. Therefore, it has been assumed that the detection of complement factor H is a viable surrogate marker for the determination of residual contaminating blood in cCSF and CBL samples.

As positive controls human pooled serum (HPS; 200-800 μg/mL complement factor H) and human pooled plasma (HPP; about 300 μg/mL complement factor H) were available.

The assay was set-up as an Elecsys-assay (Roche Diagnostics GmbH, Mannheim, Germany). The respective calibration curve is shown in FIG. 7. The working range of this assay was between 7.8 μg/mL and 2000 μg/mL.

| sample | | | μg/mL |
|---|---|---|---|
| positive | human pooled plasma | | 322 |
| control | human pooled serum | | 324 |
| 1 | cynomolgus pooled serum | | 1.4 |
| 2 | cynomolgus pooled plasma 1 | | 1.4 |
| 3 | cynomolgus pooled plasma 2 | | 1.4 |
| 4 | animal 01 | HH8061813 | 1.2 |
| 5 | animal 02 | HH8061813 | 1.2 |
| 6 | animal 12 | HH7061813 | 1.2 |
| 7 | animal 07 | HH7061813 | 1.0 |
| 8 | animal 08 | HH7061813 | 1.0 |

| | | |
|---|---|---|
| 9 | cynomolgus pooled plasma 1 | 1.1 |
| 10 | cynomolgus pooled plasma 2 | 1.3 |
| 11 (replica | cynomolgus pooled serum | 1.5 |
| of 1) | | |
| negative | cynomolgus brain lysate animal 1 | blank value |
| control | cynomolgus brain lysate animal 2 | blank value |
| | cynomolgus cerebrospinal fluid | blank value |

Thus, it has been found that the determination of complement factor H is not suited as surrogate marker for residual contaminating blood as the assay is not sensitive enough.

Determination of alpha-2-macroglobulin

Different publications indicated that in cynomolgus cerebrospinal fluid (liquor cerebrospinalis) as well as cynomolgus brain lysates no α2-macroglobulin is present, wherein it is common in non-CSF or non-brain tissue (1500-2000 μg/mL). Therefore, it has been assumed that the detection of α2-macroglobulin is a viable surrogate marker for the determination of residual contaminating blood in cCSF and CBL samples.

The assay principle of the ELISA-assay for the determination α2-macroglobulin is shown in FIG. 8 and a respective calibration curve in FIG. 9.

The assay had a working range from 0.62 ng/ml (LLOQ) to 39 ng/ml (ULOQ).

| | | |
|---|---|---|
| human pooled serum | 2.8 | g/l |
| cynomolgus pooled serum 1 | 36 | ng/ml |
| cynomolgus pooled serum 2 | 44 | ng/ml |
| cynomolgus brain lysate sample | 0 | ng/ml |

The expected values for human serum and plasma could be confirmed whereas in cynomolgus pooled serum only 1/25,000 of the expected amount could be detected. Thus, this value is too low to be quantified in diluted form in cynomolgus cerebrospinal fluid and brain lysates. Thus, the determination of α2-macroglobulin is not suited as surrogate marker.

### Determination of Complement Component 5a (C5a)

Different publications indicated that in cynomolgus cerebrospinal fluid (liquor cerebrospinalis) as well as cynomolgus brain lysates no complement component 5a is present, wherein it is common in non-CSF or non-brain tissue (60-110 μg/mL in human serum). Therefore, it has been assumed that the detection of complement component C5a is a viable surrogate marker for the determination of residual contaminating blood in cCSF and CBL samples.

Like for α2-macroglobulin an ELISA has been set-up with a murine anti-human C5a antibody as capture antibody and a biotinylated murine anti-human C5a antibody as detection antibody, whereby both antibodies bind to non-interfering epitopes on human C5a. A respective calibration curve is shown in FIG. 10.

The assay had a working range from 0.03 ng/mL (LLOQ) to 2 ng/ml (ULOQ).

| | final | |
|---|---|---|
| | concentration | |
| | in matrix | |
| human pooled serum | 57 | μg/mL |
| human pooled plasma | 24.5 | μg/mL |
| cynomolgus pooled serum 1 | 52 | μg/mL |
| cynomolgus pooled serum 2 | 70 | μg/mL |
| cynomolgus brain lysate | 11 | μg/mL |

Thus, it has been found that C5a can be determined in CBL samples.

Thus, the determination of C5a is not suited as surrogate marker.

### Use of Magnevist®

Magnevist® (gadopentetat-dimeglumin) is a MRT-imaging agent. It was assumed that Magnevist® will not passage the blood-brain-barrier.

A pharmacokinetic study showed that up to 15 minutes only the measured brain concentration represents the blood compartment correctly. After this time, there is diffusion of Magnevist® into the brain tissue, confounding the applied correction. The respective time-course is shown in FIG. 11. At the 5-minute time point, plasma volume is estimated as 14.1 μL/g brain.

Thus, this approach cannot be sued with perfusion, as the time taken for perfusion would lead to a diffusion of Magnevist across the blood-brain-barrier—confounding the residual blood correction.

Determination of Cynomolgus IgG in Cynomolgus Cerebrospinal Fluid (cCSF)

Different publications indicated that in cynomolgus cerebrospinal fluid (liquor cerebrospinalis) only minor amounts of cynomolgus IgG are present. Therefore, it has been assumed that the detection of total Ig in cCSF is a viable surrogate marker for the direct determination of transported therapeutic antibody. Therefore, a bridging ELISA was set up as shown in FIG. 12. To exclude matrix effects a human IgG-depleted cCSF was generated by incubating the cCSF with anti-human CH1/kappa antibody bound to magnetic beads.

The respective calibration curves with buffer and human IgG-depleted cCSF are shown in FIG. 13. It can be seen that no matrix effect occurs.

The assay had a working range of from 120 ng/ml to 7.2 ng/mL IgG.

Using this assay it has been found that in cynomolgus pooled plasma samples (CPP) about 11-19 mg/mL IgG could be detected, whereas in cCSF samples substantial amounts of about 4-18 μg/mL cynomolgus IgG could be detected.

The following examples, sequences and figures are provided to aid the understanding of the present invention, the true scope of which is set forth in the appended claims. It is understood that modifications can be made in the procedures set forth without departing from the spirit of the invention.

### Description of the Sequences

| SEQ | |
|---|---|
| ID NO | description |
| 01 | human Aβ42 |
| 02 | (G4S)4 linker |
| 03 | (G4S)6G2 linker |
| 04 | mAh 0012 |
| 05 | mAh 0012 |
| 06 | mAh 0012 |
| 07 | mAh 0012 |
| 08 | mAh 0015 |
| 09 | mAh 0015 |
| 10 | mAh 0015 |
| 11 | mAh 0015 |
| 12 | mAh 0020 |
| 13 | mAh 0020 |
| 14 | mAh 0020 |
| 15 | mAh 0024 |
| 16 | mAh 0024 |
| 17 | mAh 0024 |
| 18 | mAh 0024 |
| 19 | A-beta binding site VH |
| 20 | A-beta binding site VL |
| 21 | transferrin receptor binding site VH |
| 22 | transferrin receptor binding site VL |
| 23 | light chain |
| 24 | heavy chain |
| 25 | light chain |
| 26 | heavy chain Fab fragment |
| 27 | anti-transferrin receptor antibody 128.1 VH |
| 28 | anti-transferrin receptor antibody 128.1 VL |
| 29 | heavy chain variable domain |
| 30 | light chain variable domain |
| 31 | anti-transferrin receptor binding site VH |
| 32 | anti-transferrin receptor binding site VL |
| 33 | the anti-transferrin receptor binding site HVR-H1 |
| 34 | the anti-transferrin receptor binding site HVR-H2 |
| 35 | the anti-transferrin receptor binding site HVR-H3 |
| 36 | the anti-transferrin receptor binding site HVR-H3 |
| 37 | the anti-transferrin receptor binding site HVR-H3 |
| 38 | the anti-transferrin receptor binding site HVR-L1 |
| 39 | the anti-transferrin receptor binding site HVR-L2 |
| 40 | the anti-transferrin receptor binding site HVR-L3 |
| 41 | heavy chain variable domain anti-CD20 antibody |
| 42 | light chain variable domain anti-CD20 antibody |
| 43 | heavy chain variable domain anti-alpha synuclein |
| | (asyn) antibody |
| 44 | light chain variable domain anti-alpha synuclein |
| | antibody |
| 45 | heavy chain variable domain anti-alpha synuclein |
| | antibody |
| 46 | light chain variable domain anti-alpha synuclein |
| | antibody |
| 47 | heavy chain variable domain anti-alpha synuclein |
| | antibody |
| 48 | light chain variable domain anti-alpha synuclein |
| | antibody |
| 49 | heavy chain variable domain anti-alpha synuclein |
| | antibody |
| 50 | light chain variable domain anti-alpha synuclein |
| | antibody |
| 51 | heavy chain variable domain anti-alpha synuclein |
| | antibody |
| 52 | light chain variable domain anti-alpha synuclein |
| | antibody |
| 53 | heavy chain variable domain anti-alpha synuclein |
| | antibody |
| 54 | light chain variable domain anti-alpha synuclein |
| | antibody |
| 55 | glucocerebrosidase |
| 56 | peptidic linker |
| 57 | peptidic linker |
| 58 | brain target |
| 59 | brain target |
| 60 | brain target |
| 61 | brain target |
| 62 | the anti-transferrin receptor binding site HVR-H1 |
| 63 | the anti-transferrin receptor binding site HVR-H2 |
| 64 | the anti-transferrin receptor binding site HVR-H3 |
| 65 | the anti-transferrin receptor binding site HVR-L1 |
| 66 | the anti-transferrin receptor binding site HVR-L3 |
| 67 | heavy chain variable region sequence DP47GS |
| 68 | light chain variable region sequence DP47GS |
| 69 | inert reference monoclonal antibody comprises a |
| | heavy chain |
| 70 | inert reference monoclonal antibody comprises a |
| | light chain |

### DESCRIPTION OF THE FIGURES

FIG. 1 Exemplary calculation for determining residual-plasma-corrected brain lysate concentration of a tmAb.

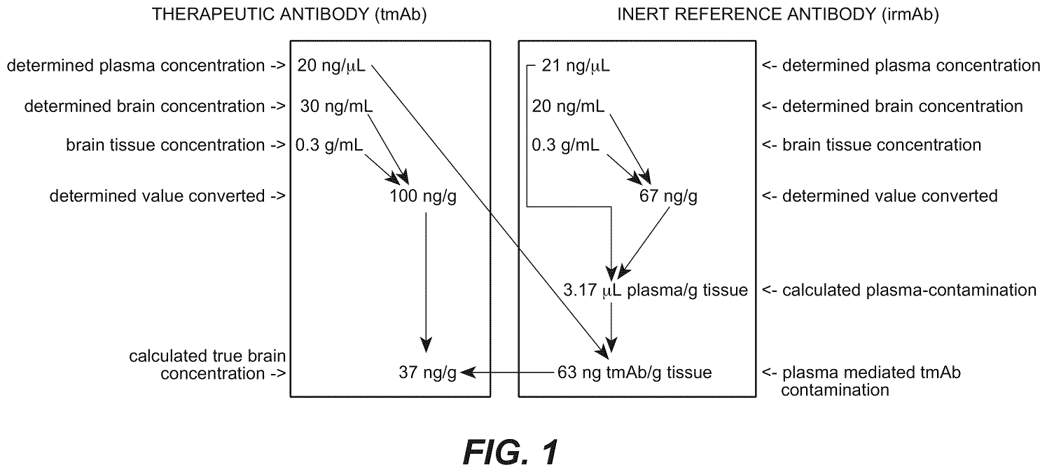

FIG. 2 Structure of the exemplary brain-shuttle construct used in the examples of the method according to the invention.

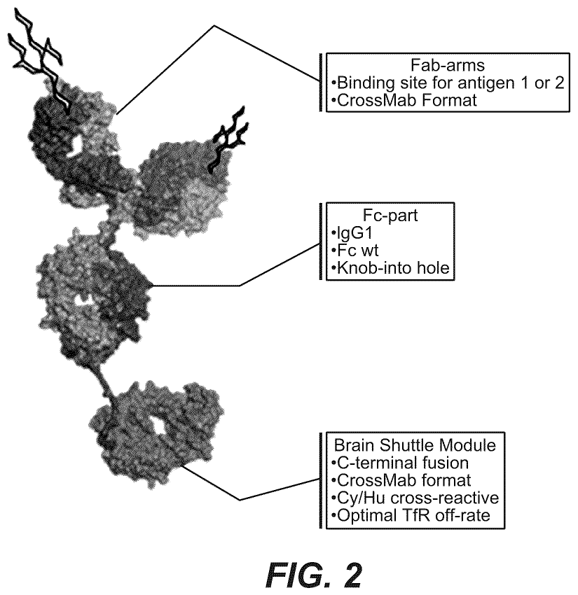

FIG. 3 Detection assay for therapeutic antibody and reference antibody used in the Examples.

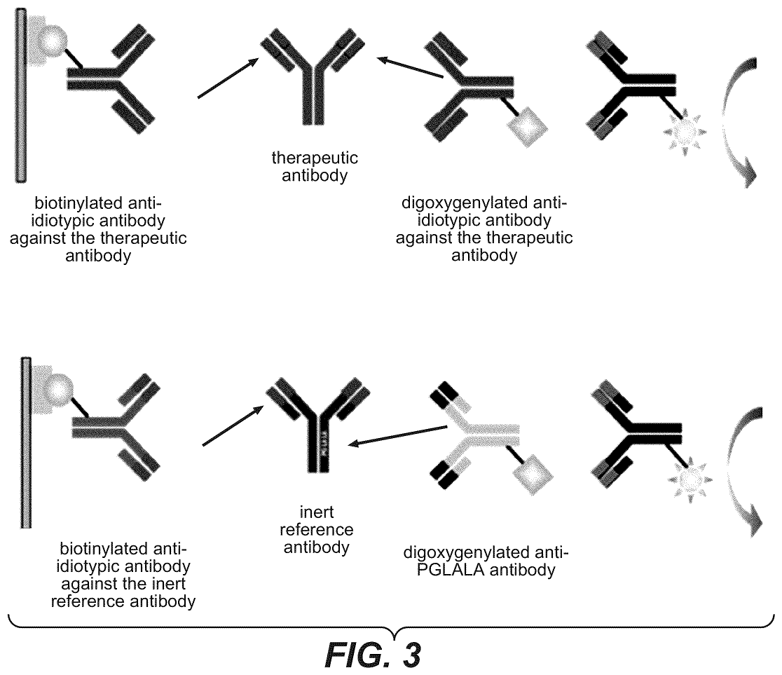

FIG. 4 Overlay of the calibration curves of the detection assay according to Example 1 for the inert reference antibody in the presence of 1% cynomolgus and mouse, respectively, brain lysate.

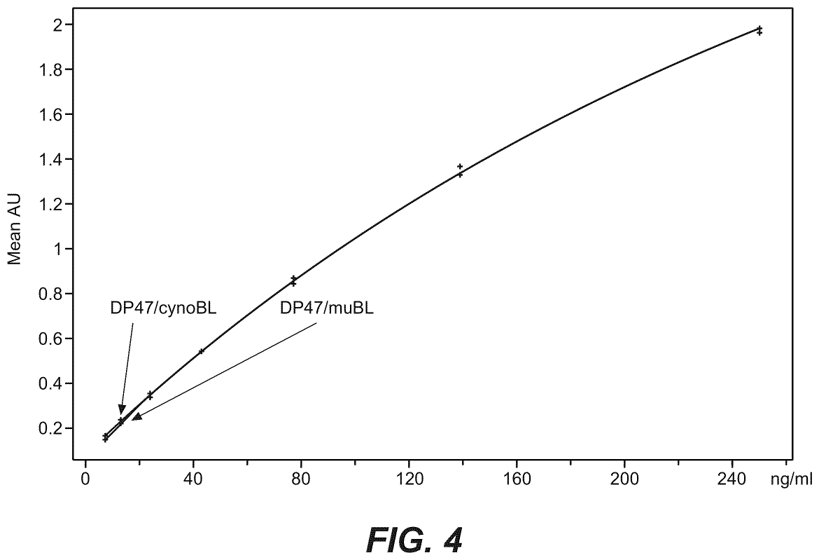

FIG. 5 Ratio of corrected concentration to non-corrected concentration of antibody 2 in mouse brain lysate.

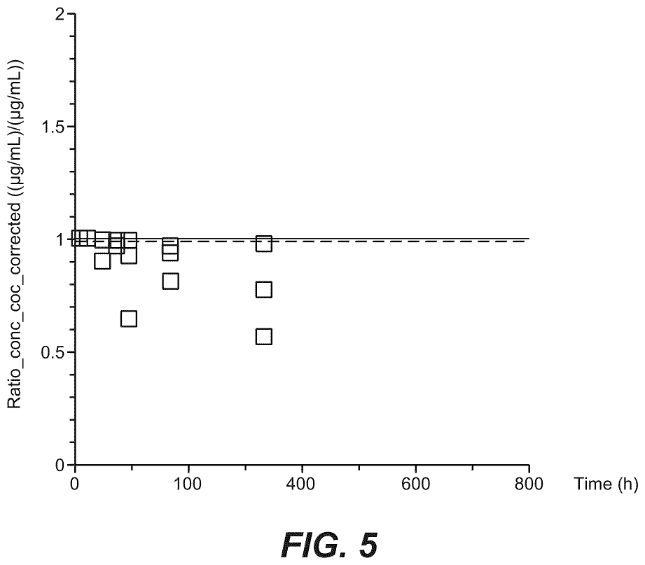

FIG. 6 ELISA for the determination of antibody 2.

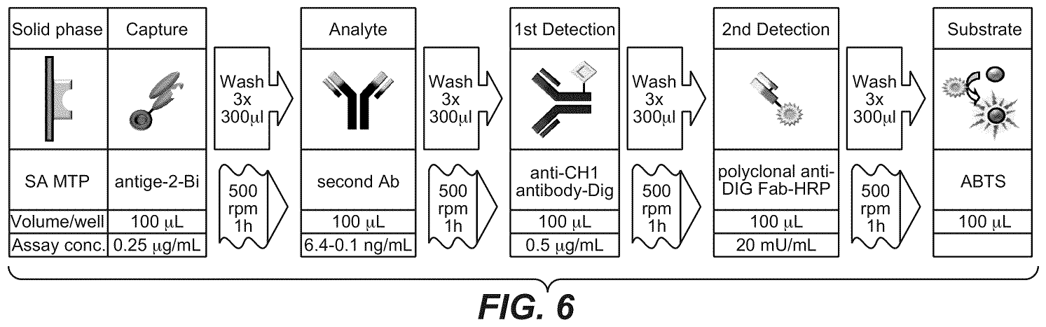

FIG. 7 Calibration curve of complement factor H Elecsys assay.

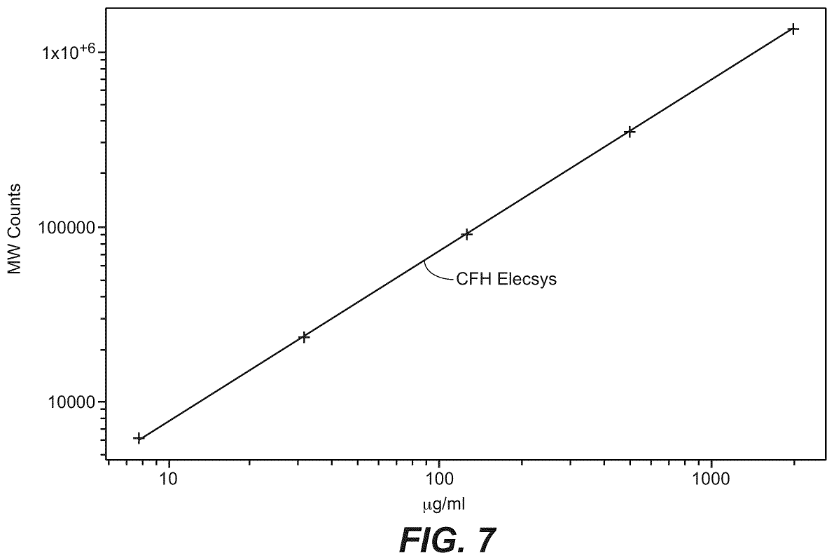

FIG. 8 ELISA for the quantification of α2-macroglobulin in cCSF. The capture antibody is a murine anti-human α2-macroglobulin antibody; the detection antibody is a biotinylated goat anti-human α2-macroglobulin antibody.

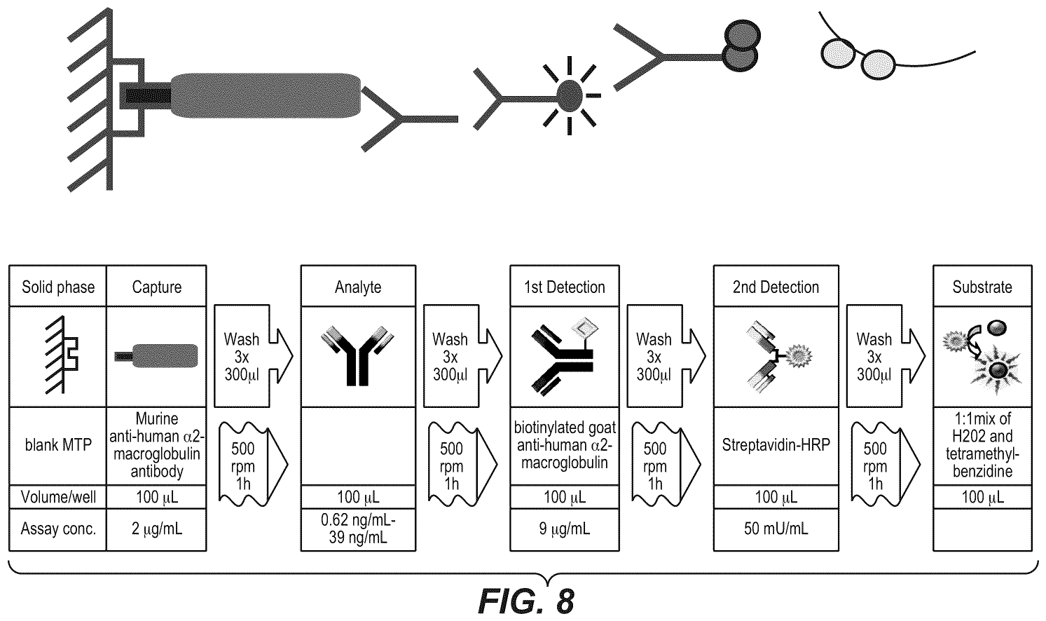

FIG. 9 Calibration curve of the ELISA of FIG. 8.

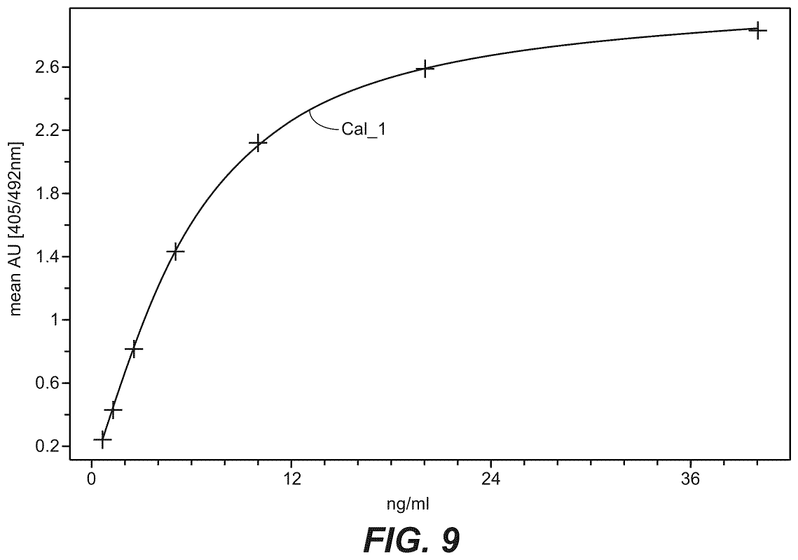

FIG. 10 Calibration curve of complement component 5a ELISA assay.

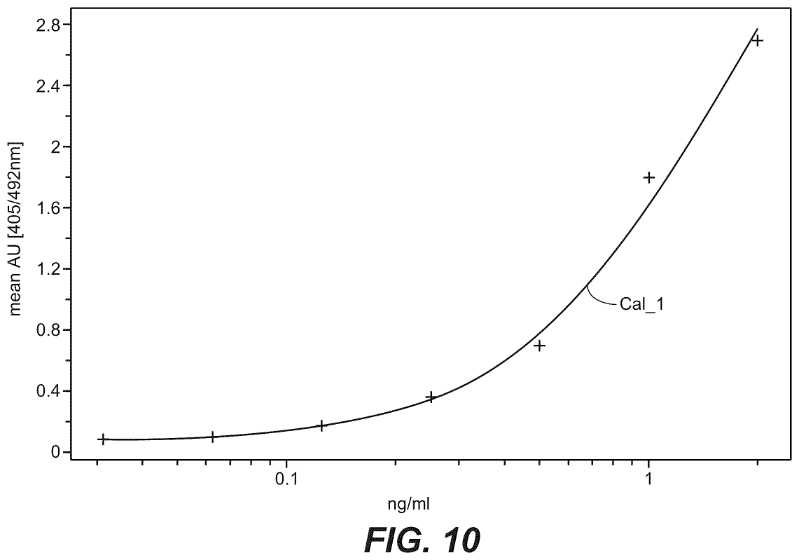

FIG. 11 Time course of Magnevist in PK study in plasma and brain tissue.

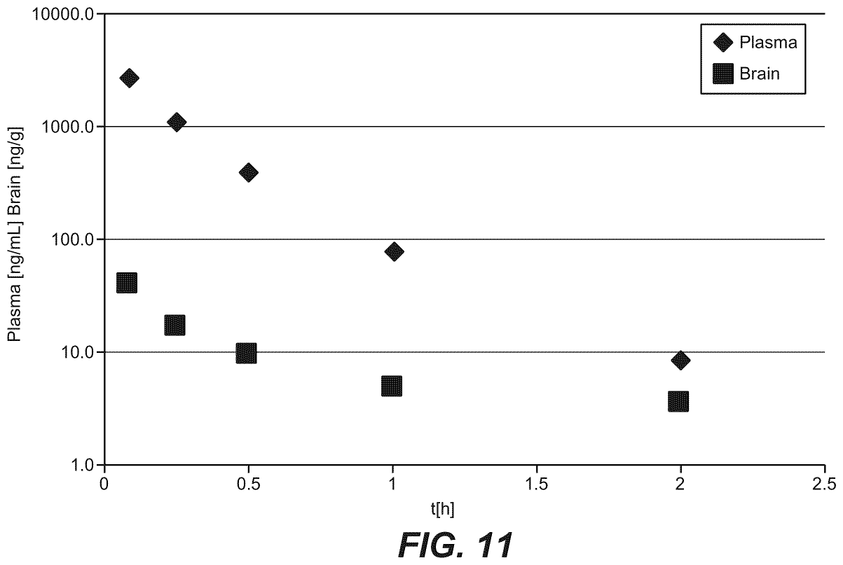

FIG. 12 ELISA for the quantification of cynomolgus IgG in cCSF. The capture mAb is an anti-cyno IgG antibody; the detection mAb is an anti-cyno IgG antibody binding to a not interference epitope with respect to the first antibody.

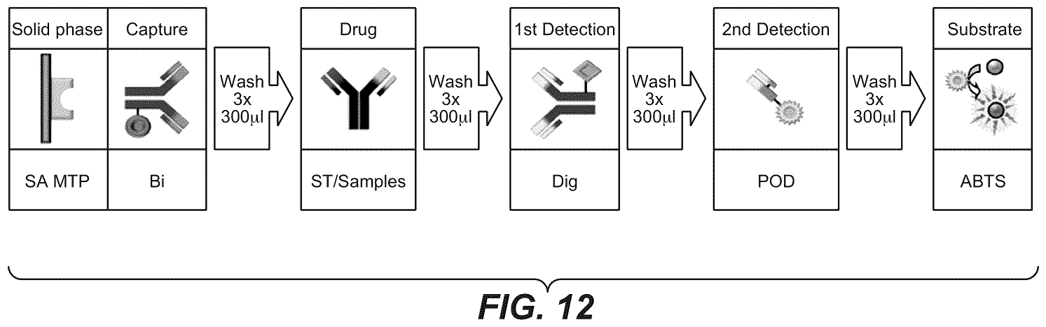

FIG. 13 Calibration curve of the ELISA of FIG. 12.

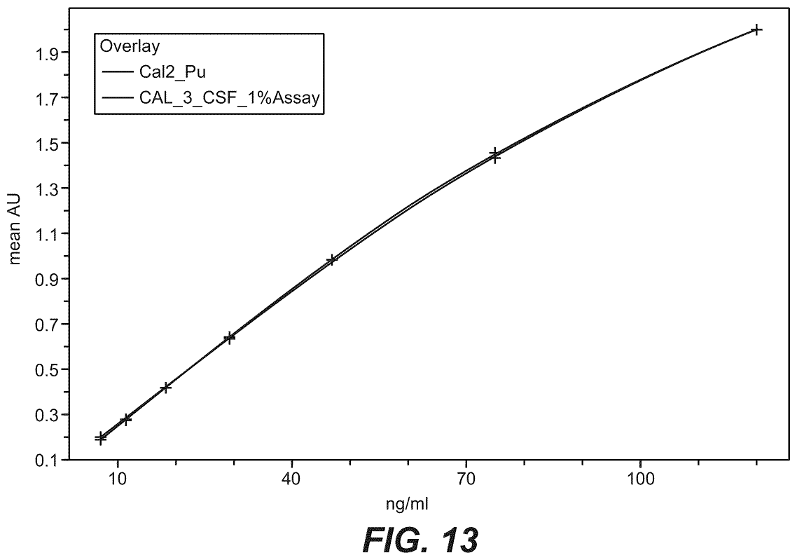

### GENERAL METHODS

### Preparation of Cynomolgus Brain Tissue Homogenates

Frozen cynomolgus/mouse brain tissue samples of 300 mg were thawed at room temperature for 2 h. 800 μL of lysis buffer, one tablet of complete protease inhibitor cocktail (Roche Diagnostics GmbH) dissolved in 50 mL Tissue Extraction Reagent I (Invitrogen), were added to the thawed brain tissue. Next, the sample was homogenized in a MagNA Lyser instrument (Roche Diagnostics) for 20 seconds at 6500 rpm. The tissue homogenate was then centrifuged for 10 minutes at 12,000 rpm using a Centrifuge 5430 (Eppendorf). Finally, the supernatant was transferred to a 1.5 mL vial for further analysis or stored at −80° C.

### Example 1

### ELISA for the Quantification of DP47GS-PGLALA in Brain Lysates

To quantify the inert reference monoclonal antibody DP47GS-PGLALA (SEQ ID NO: 69 and 70) in cynomolgus brain lysate samples a serial sandwich enzyme linked immunosorbent assay (ELISA) was used. In the ELISA procedure, all samples and controls are subjected to an initial 1:100 pre-dilution in Assay Diluent to the desired 1% final assay concentration.

Capture Antibody (anti-DP47GS antibody, biotinylated), diluted Calibrators (DP47GS-PGLALA) as well as diluted Quality controls and samples, detection reagent (anti-PGLALA antibody clone M-1.7.24, digoxygenylated) and anti-digoxygenin-antibody-POD-conjugate are added successively to a streptavidin coated microtiter plate (SA-MTP). The reagents were incubated for 1 hour on a MTP shaker at 500 rpm and after each step the MTP was washed three times with 300 μL washing buffer (1×PBS, 0.05% Tween) and residual fluids were removed. After that, the formed immobilized immune complexes were visualized by addition of ABTS solution, a horseradish POD substrate, which was converted to a colored reaction product. Finally, the color intensity was photometrically determined (absorption at 405 nm-490 nm reference wavelength). The signal is proportional to the analyte concentration in the brain lysate sample. The quantification of DP47GS-PGLALA was performed by back calculation of the absorbance values using the corresponding calibration curve with a non-linear 4-parameter Wiemer-Rodbard curve fitting function with weighting.

| | | brain lysate | assay |
|---|---|---|---|
| | | concentration | concentration |
| | | DP47GS- | DP47GS- |
| | | PGLALA | PGLALA |
| Quality control | Calculation | [ng/mL] | [ng/mL] |
| ULQC | highest | 250 | 2.5 |
| Upper limit of | calibrator | | |
| quantification | | | |
| HQC | highest | 185 | 1.85 |
| High range | calibrator × | | |
| | 0.75 | | |
| MQC | geometric | 80 | 0.80 |
| Medium range | mean of | | |
| | HQC and LQC | | |
| LQC | LLQC × 3 | 25 | 0.25 |
| Low range | | | |
| LLQC | lowest | 8 | 0.08 |
| Lower limit of | calibrator | | |
| quantification | | | |

Coating with capture reagent is achieved by pipetting 100 μL of a solution comprising 500 ng/mL biotinylated anti-DP47GS antibody into each SA-MTP well. Thereafter the MTP is covered with adhesive cover foil and incubated for 1 hour on a MTP shaker (500 rpm). The supernatant is removed and each well of the MTP is washed three times with 300 μL washing buffer (PBS, 0.05% Tween). Residual washing buffer is carefully removed.

Then 100 μl of the respective calibrators, quality controls and samples to the designated wells of the coated MTP. Thereafter the MTP is covered with adhesive cover foil and incubated for 1 hour on a MTP shaker (500 rpm). The supernatant is removed and each well of the MTP is washed three times with 300 μL washing buffer (PBS, 0.05% Tween). Residual washing buffer is carefully removed.

Then 100 μL of digoxygenylated anti-PGLALA antibody clone M-1.7.24 at a concentration of 125 ng/ml is added to each MTP well. Thereafter the MTP is covered with adhesive cover foil and incubated for 1 hour on a MTP shaker (500 rpm). The supernatant is removed and each well of the MTP is washed three times with 300 μL washing buffer (PBS, 0.05% Tween). Residual washing buffer is carefully removed.

The 100 μL of an anti-digoxygenin antibody-POD-conjugate at a concentration of 50 mU/mL is added to each MTP well. Thereafter the MTP is covered with adhesive cover foil and incubated for 1 hour on a MTP shaker (500 rpm). The supernatant is removed and each well of the MTP is washed three times with 300 μL washing buffer (PBS, 0.05% Tween). Residual washing buffer is carefully removed.

Then 100 μL ABTS solution is added to each MTP well. The optical density is measured until the average signal of the duplicates of Calibrator sample 1 reaches 1.8-2.2 AU at a measuring wavelength of 405 nm (reference wavelength 490 nm).

### Example 2

### ELISA for the Quantification of Cynomolgus IgG in CSF

To quantify cynomolgus IgG in cynomolgus cerebrospinal fluid a serial sandwich enzyme linked immunosorbent assay (ELISA) was used. In the ELISA procedure, all samples and controls are subjected to an initial pre-dilution in Assay Diluent to the desired 1% final assay concentration.

Capture Antibody (anti-cynomolgus IgG antibody 1; epitope 1; biotinylated), diluted Calibrators as well as diluted Quality controls and samples, detection reagent (anti-cynomolgus IgG antibody 2; epitope 2, not interfering with epitope 1; digoxygenylated) and anti-digoxygenin-antibody-POD-conjugate are added successively to a streptavidin coated microtiter plate (SA-MTP). The reagents were incubated for 1 hour on a MTP shaker at 500 rpm and after each step the MTP was washed three times with 300 μL washing buffer (1×PBS, 0.05% Tween) and residual fluids were removed. After that, the formed immobilized immune complexes were visualized by addition of ABTS solution, a horseradish POD substrate, which was converted to a colored reaction product. Finally, the color intensity was photometrically determined (absorption at 405 nm-490 nm reference wavelength). The signal is proportional to the analyte concentration in the brain lysate sample. The quantification of cynomolgus IgG was performed by back calculation of the absorbance values using the corresponding calibration curve with a non-linear 4-parameter Wiemer-Rodbard curve fitting function with weighting.

| | | cCSF | assay |
|---|---|---|---|
| | | concentration | concentration |
| | | cynomolgus IgG | cynomolgus IgG |
| Quality control | Calculation | [ng/mL] | [ng/mL] |
| ULQC | highest | 12000 | 120 |
| Upper limit of | calibrator | | |
| quantification | | | |
| HQC | highest | 9000 | 90 |
| High range | calibrator × | | |
| | 0.75 | | |
| MQC | geometric | 45000 | 45 |
| Medium range | mean of | | |
| | HQC and LQC | | |
| LQC | LLQC × 3 | 2200 | 22 |
| Low range | | | |
| LLQC | lowest | 720 | 7.2 |
| Lower limit of | calibrator | | |
| quantification | | | |

Coating with capture reagent is achieved by pipetting 100 μL of a solution comprising 250 ng/mL biotinylated anti-cynomolgus IgG antibody 1 into each SA-MTP well. Thereafter the MTP is covered with adhesive cover foil and incubated for 1 hour on a MTP shaker (500 rpm). The supernatant is removed and each well of the MTP is washed three times with 300 μL washing buffer (PBS, 0.05% Tween). Residual washing buffer is carefully removed.

Then 100 μl of the respective calibrators, quality controls and samples to the designated wells of the coated MTP Thereafter the MTP is covered with adhesive cover foil and incubated for 1 hour on a MTP shaker (500 rpm). The supernatant is removed and each well of the MTP is washed three times with 300 μL washing buffer (PBS, 0.05% Tween). Residual washing buffer is carefully removed.

Then 100 μL of digoxygenylated anti-cynomolgus antibody 2 at a concentration of 250 ng/ml is added to each MTP well. Thereafter the MTP is covered with adhesive cover foil and incubated for 1 hour on a MTP shaker (500 rpm). The supernatant is removed and each well of the MTP is washed three times with 300 μL washing buffer (PBS, 0.05% Tween). Residual washing buffer is carefully removed.

The 100 μL of an anti-digoxygenin antibody-POD-conjugate at a concentration of 25 mU/mL is added to each MTP well. Thereafter the MTP is covered with adhesive cover foil and incubated for 1 hour on a MTP shaker (500 rpm). The supernatant is removed and each well of the MTP is washed three times with 300 μL washing buffer (PBS, 0.05% Tween). Residual washing buffer is carefully removed.

Then 100 μL ABTS solution is added to each MTP well. The optical density is measured until the average signal of the duplicates of Calibrator sample 1 reaches 1.8-2.2 AU at a measuring wavelength of 405 nm (reference wavelength 490 nm).

### Example 3

### Production of Brain Tissue Lysates

First, the lysis buffer was freshly prepared according to the manufacturer's instructions (Invitrogen; tissue extraction reagent I; Cat.-No. FNN0071). Per 50 ml of lysis buffer 1 tables of Complete is added (Roche Diagnostics GmbH, Mannheim, Germany; Cat.-No. 11697498001).

Second, to the brain tissue sample, approx. 100-300 mg, between 600 μL and 800 μL were lysis buffer is added. Optionally MagNA Lyser Green Beads are added.

Third, the samples were placed for 20 sec. at 6500 rpm in the MagNA Lyser (Roche Diagnostics GmbH. Mannheim, Germany).

Fourth, after incubation in the MagNA Lyser the samples are centrifuged for 10 min. at 12,000 rpm (Eppendorf Centrifuge 5430).

Fifth, the supernatant (500-700 μL) was recovered and stored at −80° C. until further analysis.

1. A method or assay for determining the concentration of a therapeutic antibody in a tissue of an experimental animal, whereby the tissue has a barrier to the blood circulation of said animal and whereby the therapeutic antibody had been administered to said experimental animal, wherein the interference from residual blood in a tissue sample of the experimental animal, which is used for determining the concentration of the therapeutic antibody in said tissue, is reduced, the method comprising the following steps
i) determining the concentration of the therapeutic antibody in a blood sample of the experimental animal,
ii) determining the concentration of the therapeutic antibody in the tissue sample of the experimental animal,
iii) determining the concentration of an inert reference antibody in the blood sample of the experimental animal,
iv) determining the concentration of the inert reference antibody in the tissue sample of the experimental animal,
v) determining the tissue concentration in the tissue sample,
and determining the concentration of the therapeutic antibody in the tissue of the experimental animal with the following formula:
$${C}_{tmAb , tissue} = \frac{{c}_{tmAb , tissue , det .}}{{c}_{tissue , sample}} - \frac{\frac{{c}_{refmAb , tissue , det .}}{{c}_{tissue , sample}}}{{c}_{refmAb , plasma . det .}} * {C}_{tmAb , plasma , det .}$$
with
C_{tmAb,plasma,det.}=concentration of the therapeutic antibody of i)
C_{tmAb,tissue,det.}=concentration of the therapeutic antibody of ii)
C_{refmAb,tissue,det.}=concentration of the therapeutic antibody of iii)
C_{refmAb,plasma,det.}=concentration of the therapeutic antibody of iv)
C_{tissue,sample}=tissue concentration of v)
whereby the inert reference antibody does not cross said barrier between the tissue and the blood circulation,
whereby the inert reference antibody had been administered i) either together with the therapeutic antibody in case the sample is to be taken within 5 minutes after the administration of the therapeutic antibody, or ii) 2 to 10 minutes prior to taking the tissue sample,
whereby the blood sample is taken directly prior to the tissue sample.

2. The Method according to claim 1, wherein the tissue is either brain tissue and the therapeutic antibody can cross the blood-brain-barrier or ocular tissue and the therapeutic antibody can cross the blood-ocular-barrier.

3. The method according to any one of claims 1 to 2, wherein the therapeutic antibody is a bispecific antibody.

4. The method according to any one of claims 1 to 3, wherein the therapeutic antibody is specifically binding to human transferrin receptor and a brain target.

5. The method according to claim 4, wherein the brain target is human CD20 or human Abeta or human alpha-synuclein or human tau or human glucocerebrosidase or human lingo-1 or human huntingtin.

6. The method according to any one of claims 1 to 5, wherein the experimental animal is selected from mouse, rat, rabbit, dog, sheep, ape, and monkey.

7. The method according to any one of claims 1 to 6, wherein the experimental animal is a non-human experimental animal with a body weight of more than 100 g and less than 15 kg.

8. The method according to any one of claims 1 to 7, wherein the experimental animal is a cynomolgus monkey.

9. The method according to any one of claims 1 to 8, wherein the inert reference antibody is a human germline antibody.

10. The method according to any one of claims 1 to 9, wherein the inert reference antibody is DP47GS.

11. The method according to any one of claims 1 to 10, wherein the inert reference antibody does not cross said barrier in detectable amounts within 15 minutes after its application.

12. The method according to claim 11, wherein the inert reference antibody does not cross said barrier in detectable amounts within 10 minutes after its application.

13. The method according to any one of claims 1 to 12, wherein the inert antibody is administered about 5 minutes prior to taking the tissue sample.

14. The method according to any one of claims 1 to 10, wherein the tissue is perfused with an aqueous solution directly after taking the blood sample and prior to taking the tissue sample.

15. The method according to any one of claims 1 to 11, wherein the determining of the concentrations is by a bridging ELISA.


SEQUENCE LISTING (70 sequences)

SEQ ID NO: 1 - protein, 42 aa, Homo sapiens [sequence omitted]

SEQ ID NO: 2 - protein, 20 aa, synthetic construct
  REGION 1..20: note = (G4S)4 linker
GGGGSGGGGSGGGGSGGGGS

SEQ ID NO: 3 - protein, 32 aa, synthetic construct
  REGION 1..32: note = (G4S)6G2 linker
GGGGSGGGGSGGGGSGGGGSGGGGSGGGGSGG

SEQ ID NO: 4 - protein, 215 aa, synthetic construct
  REGION 1..215: note = 0012-LC1
DIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 455 aa, synthetic construct
  REGION 1..455: note = 0012-HC1
QVELVESGGGLVQPGGSLRLSCAASGFTFSSYAMSWVRQAPGKGLEWVSAINASGTRTYYADSVKGRFTISRDNSKNTLYLQMNSLRAEDTAVYYCARGKGNTHKPYGYVRYFDVWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 6 - protein, 229 aa, synthetic construct
  REGION 1..229: note = 0012-LC2
QSMQESGPGLVKPSQTLSLTCTVSGFSLSSYAMSWIRQHPGKGLEWIGYIWSGGSTDYASWAKSRVTISKTSTTVSLKLSSVTAADTAVYYCARRYGTSYPDYGDASGFDPWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 688 aa, synthetic construct
  REGION 1..688: note = 0012-HC2
QVELVESGGGLVQPGGSLRLSCAASGFTFSSYAMSWVRQAPGKGLEWVSAINASGTRTYYADSVKGRFTISRDNSKNTLYLQMNSLRAEDTAVYYCARGKGNTHKPYGYVRYFDVWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSAIQLTQSPSSLSASVGDRVTITCRASQSISSYLAWYQQKPGKAPKLLIYRASTLASGVPSRFSGSGSGTDFTLTISSLQPEDFATYYCQQNYASSNVDNTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 8 - protein, 215 aa, synthetic construct; identical to SEQ ID NO: 4
  REGION 1..215: note = 0015-LC1

SEQ ID NO: 9 - protein, 455 aa, synthetic construct; identical to SEQ ID NO: 5
  REGION 1..455: note = 0015-HC1

SEQ ID NO: 10 - protein, 215 aa, synthetic construct
  REGION 1..215: note = 0015-LC2
AIQLTQSPSSLSASVGDRVTITCRASQSISSYLAWYQQKPGKAPKLLIYRASTLASGVPSRFSGSGSGTDFTLTISSLQPEDFATYYCQQNYASSNVDNTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 11 - protein, 702 aa, synthetic construct
  REGION 1..702: note = 0015-HC2
QVELVESGGGLVQPGGSLRLSCAASGFTFSSYAMSWVRQAPGKGLEWVSAINASGTRTYYADSVKGRFTISRDNSKNTLYLQMNSLRAEDTAVYYCARGKGNTHKPYGYVRYFDVWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSQSMQESGPGLVKPSQTLSLTCTVSGFSLSSYAMSWIRQHPGKGLEWIGYIWSGGSTDYASWAKSRVTISKTSTTVSLKLSSVTAADTAVYYCARRYGTSYPDYGDASGFDPWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 215 aa, synthetic construct; identical to SEQ ID NO: 4
  REGION 1..215: note = 0020-LC1

SEQ ID NO: 13 - protein, 457 aa, synthetic construct
  REGION 1..457: note = 0020-HC1
QVELVESGGGLVQPGGSLRLSCAASGFTFSSYAMSWVRQAPGKGLEWVSAINASGTRTYYADSVKGRFTISRDNSKNTLYLQMNSLRAEDTAVYYCARGKGNTHKPYGYVRYFDVWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKG

SEQ ID NO: 14 - protein, 947 aa, synthetic construct
  REGION 1..947: note = 0020-HC2
QVELVESGGGLVQPGGSLRLSCAASGFTFSSYAMSWVRQAPGKGLEWVSAINASGTRTYYADSVKGRFTISRDNSKNTLYLQMNSLRAEDTAVYYCARGKGNTHKPYGYVRYFDVWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSAIQLTQSPSSLSASVGDRVTITCRASQSISSYLAWYQQKPGKAPKLLIYRASTLASGVPSRFSGSGSGTDFTLTISSLQPEDFATYYCQQNYASSNVDNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGQSMQESGPGLVKPSQTLSLTCTVSGFSLSSYAMSWIRQHPGKGLEWIGYIWSGGSTDYASWAKSRVTISKTSTTVSLKLSSVTAADTAVYYCARRYGTSYPDYGDASGFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 15 - protein, 215 aa, synthetic construct
  REGION 1..215: note = 0024-LC1
DIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 455 aa, synthetic construct
  REGION 1..455: note = 0024-HC1
QVELVESGGGLVQPGGSLRLSCAASGFTFSSYAMSWVRQAPGKGLEWVSAINASGTRTYYADSVKGRFTISRDNSKNTLYLQMNSLRAEDTAVYYCARGKGNTHKPYGYVRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 17 - protein, 215 aa, synthetic construct; identical to SEQ ID NO: 10
  REGION 1..215: note = 0024-LC2

SEQ ID NO: 18 - protein, 702 aa, synthetic construct
  REGION 1..702: note = 0024-HC2
QVELVESGGGLVQPGGSLRLSCAASGFTFSSYAMSWVRQAPGKGLEWVSAINASGTRTYYADSVKGRFTISRDNSKNTLYLQMNSLRAEDTAVYYCARGKGNTHKPYGYVRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSQSMQESGPGLVKPSQTLSLTCTVSGFSLSSYAMSWIRQHPGKGLEWIGYIWSGGSTDYASWAKSRVTISKTSTTVSLKLSSVTAADTAVYYCARRYGTSYPDYGDASGFDPWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 126 aa, Homo sapiens [sequence omitted]

SEQ ID NO: 20 - protein, 108 aa, Homo sapiens [sequence omitted]

SEQ ID NO: 21 - protein, 122 aa, synthetic construct
  REGION 1..122: note = 299-023 VH humanization variant_DASG
QSMQESGPGLVKPSQTLSLTCTVSGFSLSSYAMSWIRQHPGKGLEWIGYIWSGGSTDYASWAKSRVTISKTSTTVSLKLSSVTAADTAVYYCARRYGTSYPDYGDASGFDPWGQGTLVTVSS

SEQ ID NO: 22 - protein, 110 aa, synthetic construct
  REGION 1..110: note = 299-009 VL humanization variant_NYA
AIQLTQSPSSLSASVGDRVTITCRASQSISSYLAWYQQKPGKAPKLLIYRASTLASGVPSRFSGSGSGTDFTLTISSLQPEDFATYYCQQNYASSNVDNTFGGGTKVEIK

SEQ ID NO: 23 - protein, 215 aa, synthetic construct; identical to SEQ ID NO: 4
  REGION 1..215: note = 0015-LC1

SEQ ID NO: 24 - protein, 455 aa, synthetic construct; identical to SEQ ID NO: 5
  REGION 1..455: note = 0015-HC1

SEQ ID NO: 25 - protein, 215 aa, synthetic construct; identical to SEQ ID NO: 10
  REGION 1..215: note = 0015-LC2

SEQ ID NO: 26 - protein, 229 aa, synthetic construct; identical to SEQ ID NO: 6
  REGION 1..229: note = 0015-Fab

SEQ ID NO: 27 - protein, 118 aa, Mus musculus
QVQLVQSGAEVKKPGASVKVSCKASGYTFTGYTMNWVRQAPGQGLEWMGRINPHNGGTDYAQKFQGRVTMTRDTSISTAYMELSRLRSDDTAVYYCARGYYYYSLDYWGQGTLVTVSS

SEQ ID NO: 28 - protein, 106 aa, Mus musculus
DIQMTQSPSSLSASVGDRVTITCRASSSIRYIHWYQQKPGKAPKRLIYDTSNLASGVPSRFSGSGSGTEFTLTISSLQPEDFATYYCHQRNSYPWTFGQGTRLEIK

SEQ ID NO: 29 - protein, 122 aa, Oryctolagus cuniculus
QSMEESGGRLVTPGTPLTLTCTVSGFSLSSYAMSWVRQAPGKGLEWIGYIWSGGSTDYASWAKGRFTISKTSTTVDLKITSPTTEDTATYFCARRYGTSYPDYGDANGFDPWGPGTLVTVSS

SEQ ID NO: 30 - protein, 110 aa, Oryctolagus cuniculus
AYDMTQTPASVEVAVGGTVTIKCQASQSISSYLSWYQQKPGQRPKLLIYRASTLASGVSSRFKGSGSGTQFTLTISGVECADAATYYCQQCYSSSNVDNTFGGGTEVVVK

SEQ ID NO: 31 - protein, 122 aa, synthetic construct; identical to SEQ ID NO: 21
  REGION 1..122: note = 299-023 VH humanization variant_DASG

SEQ ID NO: 32 - protein, 110 aa, synthetic construct; identical to SEQ ID NO: 22
  REGION 1..110: note = 299-009 VL humanization variant_NYA

SEQ ID NO: 33 - protein, 7 aa, Oryctolagus cuniculus
GFSLSSY

SEQ ID NO: 34 - protein, 5 aa, Oryctolagus cuniculus
WSGGS

SEQ ID NO: 35 - protein, 17 aa, Oryctolagus cuniculus
RYGTSYPDYGDANGFDP

SEQ ID NO: 36 - protein, 17 aa, synthetic construct
  REGION 1..17: note = 299-000 HVR-H3 DASG
RYGTSYPDYGDASGFDP

SEQ ID NO: 37 - protein, 17 aa, synthetic construct
  REGION 1..17: note = 299-000 HVR-H3 DAQG
RYGTSYPDYGDAQGFDP

SEQ ID NO: 38 - protein, 11 aa, synthetic construct
  REGION 1..11: note = 299-000 HVR-L1 RAA
RASQSISSYLA

SEQ ID NO: 39 - protein, 7 aa, Oryctolagus cuniculus
RASTLAS

SEQ ID NO: 40 - protein, 12 aa, synthetic construct
  REGION 1..12: note = 299-000 HVR-L3 NYA
QQNYASSNVDNT

SEQ ID NO: 41 - protein, 119 aa, synthetic construct
  REGION 1..119: note = anti-CD20 antibody VH
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 42 - protein, 112 aa, synthetic construct
  REGION 1..112: note = anti-CD20 antibody VL
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 43 - protein, 116 aa, synthetic construct
  REGION 1..116: note = humanized anti-alpha synuclein acntibody 9E4 VH
EVQLVESGGGLVQPGGSLRLSCAASGFTFSNYGMSWVRQAPGKGLEWVASISSGGGSTYYPDNVKGRFTISRDDAKNSLYLQMNSLRAEDTAVYYCARGGAGIDYWGQGTLVTVSS

SEQ ID NO: 44 - protein, 113 aa, synthetic construct
  REGION 1..113: note = humanized anti-alpha synuclein acntibody 9E4 VL
DIQMTQSPSSLSASVGDRVTITCKSIQTLLYSSNQKNYLAWFQQKPGKAPKLLIYWASIRKSGVPSRFSGSGSGTDFTLTISSLQPEDLATYYCQQYYSYPLTFGGGTKLEIK

SEQ ID NO: 45 - protein, 115 aa, Mus musculus
QVQLQQSGAELAKPGTSVQMSCKASGYTFTNYWMNWIKARPGQGLEWIGATNPNNGYTDYNQRFKDKAILTADKSSNTAYMHLSSLTSEDSAVYFCASGGHLAYWGQGTVVTVSA

SEQ ID NO: 46 - protein, 112 aa, Mus musculus
DVVMTQIPLYLSVSPGDQASISCRSSQSLFHSKGNTYLHWYLQKPGQSPKLLINRVSNRFSGVPDRFSGSGSGTDFTLKISGVEAEDLGVYFCSQSAHVPWTFGGGTKLEIR

SEQ ID NO: 47 - protein, 115 aa, Mus musculus
VQLQQSGPELVKPGTSVKISCKASGYSFTSYYIHWVKQSPGQGLEWIGWIYPGSGNTKYSEKFKGKATLTADTSSSTAYMQLSSLTSEDSAVYFCARDGCYGFAYWGQGTLVTVS

SEQ ID NO: 48 - protein, 111 aa, Mus musculus
DIVLTQSPASLAVSLGQRATISCKASQSVDYDGDSYMNWYQQKPGQPPKFLICAASNLESGIPARFSGSGSGTDFTLNIHPVEEEDAATYYCQQSNEDPFTFGSGTKLEIK

SEQ ID NO: 49 - protein, 115 aa, Oryctolagus cuniculus
QSVEESGGRLVTPGTPLTLTCTVSGFSINSYAMIWVRQAPGEGLEWIGVIYPSGNTYYANWAKGRFTVSRTSTTVDLKITSPTTEDTATYFCARRDGTDKTFNIWGPGTLVTVSL

SEQ ID NO: 50 - protein, 112 aa, Oryctolagus cuniculus
QVLTQTPSPVSAAVGGTVTINCQASQNVYGDNYLAWYQQKPGQPPKLLIYEASKLASGVPPRFSGSGSGTQFTLTISDVQCDDAATYYCQGEFLCTTSDCFTFGGGTGVVVR

SEQ ID NO: 51 - protein, 119 aa, Oryctolagus cuniculus
QSVEESGGRLVTPGTPLTLTCTVSGIDLSRYAMSWVRQAPGKGLEWIGVINSSGATYYASWAKGRFTISETSTTVELKITSPTTEDTATYFCARWTYDDYGDFQGFNIWGPGTLVTVSL

SEQ ID NO: 52 - protein, 111 aa, Oryctolagus cuniculus
AVLTQTPSPVSAAVGGTVTISCQSSQSVYNNNDLAWYQQKPGQPPKLLIYRASKLASGVPSRFKGSGSGTQFTLTISGVQCDDAATYYCLGGYDDDADMGAFGGGTEVVVK

SEQ ID NO: 53 - protein, 111 aa, synthetic construct
  REGION 1..111: note = heavy chain variable domain
QSLEESGGRLVTPGTPLTLTCTVSGIDLSRDTMIWVRQAPGEGLEWIGSIYTDSGNTWYASWVKGRFTISKTSSTTVDLRITSPTTEDTATYFCARNFSVWGPGTLVTVSL

SEQ ID NO: 54 - protein, 112 aa, synthetic construct
  REGION 1..112: note = light chain variable domain
QVLTQTPSPVSAAVGGTVTINCQASQSVYNSDRLAWFQQMRGQPPKLLIYDVSKLASGVPSRFSGSGSGTQFTLTISDVQCDDAATYYCLGGYDCSSAECNVFGGGTEVVVK

SEQ ID NO: 55 - protein, 536 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 56 - protein, 20 aa, synthetic construct
  REGION 1..20: note = peptidic linker
GGGGSGGGGSGGGGSGGGGS

SEQ ID NO: 57 - protein, 32 aa, synthetic construct; identical to SEQ ID NO: 3
  REGION 1..32: note = peptidic linker

SEQ ID NO: 58 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 59 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 60 - protein, 441 aa, Homo sapiens
  SITE 422: note = MISC_FEATURE - X=phosphoserine
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDXPQLATLADEVSASLAKQGL

SEQ ID NO: 61 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 62 - protein, 5 aa, Oryctolagus cuniculus
SYAMS

SEQ ID NO: 63 - protein, 16 aa, Oryctolagus cuniculus
YIWSGGSTDYASWAKG

SEQ ID NO: 64 - protein, 16 aa, synthetic construct
  REGION 1..16: note = 299-000 HVR-H2 Kabat G65S
YIWSGGSTDYASWAKS

SEQ ID NO: 65 - protein, 11 aa, Oryctolagus cuniculus
QASQSISSYLS

SEQ ID NO: 66 - protein, 12 aa, Oryctolagus cuniculus
QQCYSSSNVDNT

SEQ ID NO: 67 - protein, 115 aa, synthetic construct
  REGION 1..115: note = DP47GS VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSS

SEQ ID NO: 68 - protein, 108 aa, synthetic construct
  REGION 1..108: note = DP47GS VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIK

SEQ ID NO: 69 - protein, 442 aa, synthetic construct
  REGION 1..442: note = DP47GS HC (Fc wt, P329G LALA)
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLS

SEQ ID NO: 70 - protein, 215 aa, synthetic construct
  REGION 1..215: note = DP47GS LC
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC